(19) United States
(12) Patent Application Publication
(10) Pub. No.: US 2024/0287593 A1
(43) Pub. Date: Aug. 29, 2024

(54) SINGLE-MOLECULE STRAND-SPECIFIC END MODALITIES

(71) Applicant: Centre for Novostics, New Territories (HK)

(72) Inventors: Yuk-Ming Dennis Lo, Hong Kong SAR (CN); Kwan Chee Chan, Hong Kong SAR (CN); Peiyong Jiang, New Territories (CN); Qing Zhou, New Territories (CN); Jing Liu, Hong Kong SAR (CN); Wenlei Peng, Hong Kong SAR (CN)

(21) Appl. No.: 18/586,250

(22) Filed: Feb. 23, 2024

Related U.S. Application Data
(60) Provisional application No. 63/447,847, filed on Feb. 23, 2023.

(51) Int. Cl. C12Q 1/6855; C12Q 1/6869; C12Q 1/6883; C12Q 1/6886

(52) U.S. Cl. CPC C12Q 1/6855; C12Q 1/6869; C12Q 1/6883; C12Q 1/6886

(57) ABSTRACT

The fragmentomic feature of which strand (3′ or 5′), if any, overhangs the other at one or both ends double-stranded cell-free DNA fragments can be used to analyze a biological sample. The amount of fragments with the 3′ strand overhanging the 5′ strand, the 5′ strand overhanging the 3′ strand, and/or strands being even (blunt) at one or both ends can be used to determine the type of DNA or a level of condition, including cancer and nuclease activity deficiencies. Embodiments described herein allow for determining the amount of these different end modalities, unlike prior techniques. The end modality information can be paired with end motifs to further analyze biological samples. Related systems are also described.

### CROSS-REFERENCES TO RELATED APPLICATION

This application is a nonprovisional of and claims the benefit of U.S. Provisional Patent Application No. 63/447,847 entitled “SINGLE-MOLECULE STRAND-SPECIFIC END MODALITIES,” filed on Feb. 23, 2023, which is herein incorporated by reference in its entirety for all purposes.

### BACKGROUND

Cell-free DNA has been proven to be particularly useful for molecular diagnostics and monitoring. The cell-free based applications include noninvasive prenatal testing (Chiu R K W et al. Proc Natl Acad Sci USA. 2008; 105:20458-63), cancer detection and monitoring (Chan K C A et al. Clin Chem. 2013; 59:211-24; Chan K C A et al. Proc Natl Acad Sci USA. 2013; 110:1876-8; Jiang P et al. Proc Natl Acad Sci USA. 2015; 112:E1317-25), transplantation monitoring (Zheng Y W et al. Clin Chem. 2012; 58:549-58) and tracing tissue of origin (Sun K et al. Proc Natl Acad Sci USA. 2015; 112:E5503-12; Chan K C A; Snyder M W et al. Cell. 2016; 164:57-68). Cell-free nucleic acid analysis approaches developed to date include those based on the analysis of single nucleotide variants (SNVs), copy number aberrations (CNAs), cell-free DNA ending positions in the human genome, or methylation markers. It would be beneficial to identify new nucleic acid analysis approaches for detection of new properties and to add accuracy to existing approaches.

### BRIEF SUMMARY

Double-stranded cell-free DNA fragments contain two terminal ends of each strand. One molecule can have four terminal ends. As the two strands are often not exactly complementary to each other, one strand may extend beyond the other strand, creating an overhang at the end. These overhangs are often repaired to form blunt ends in analysis, which will change the information of terminal ends of cell-free DNA fragments. This document describes how the native information of terminal ends can be obtained from each cell-free DNA fragment and may be used in analysis.

This method may concurrently assess the original 5′-, 3′-end motifs of the Watson and Crick strands, as well as the related jaggedness at single-base resolution. In some embodiments, the entire fragmentomic features from a cfDNA molecule can be analyzed accurately, including but not limited to, 5′ protruding jagged end, 3′ protruding jagged end, 5′ receded jagged end, 3′ receded jagged end, end motif of the protruding jagged end, end motif of the receded jagged ends, genomic coordinates of fragment ends, fragment sizes, methylation-associated cfDNA fragmentomic features, as well as their combinations. In some embodiments, the end motif could be defined by one or more nucleotides across positions nearby an end of a molecule. The end motif may be defined by one or more nucleotides in a reference genome surrounding the genomic locus to which the end of a fragment is aligned. In other embodiments, the jagged end could be defined by the protruding single-strand DNA at the end of the DNA fragment. The jagged end can be separated into different groups according to the length and/or strand of the protruding single-strand DNA.

In some embodiments, different fragmentomic features from one DNA fragment can be combined. In some embodiments, the combined fragmentomic features can be used for the detection or monitoring of cancer or other diseases. In other embodiments, combined fragmentomic features can be used for noninvasive prenatal testing.

A better understanding of the nature and advantages of embodiments of the present invention may be gained with reference to the following detailed description and the accompanying drawings.

### BRIEF DESCRIPTION OF THE DRAWINGS

FIG. 1 shows a schematic overview of the concurrent analysis of single-molecule end modalities on a single-molecule real-time sequencing platform according to embodiments of the present invention.

FIGS. 2A and 2B show a schematic overview of the concurrent analysis of single-molecule end modalities on a next generation sequencing (NGS) platform (e.g., Illumina platform) according to embodiments of the present invention.

FIG. 3 is a flowchart of an example process of analyzing a biological sample according to embodiments of the present invention.

FIG. 4 is a flowchart of an example process of analyzing a biological sample according to embodiments of the present invention.

FIGS. 5A and 5B show the frequencies of different jagged ends according to embodiments of the present invention.

FIG. 6A shows a graph of the overall size distribution of plasma DNA samples from a healthy subject and an HCC subject according to embodiments of the present invention.

FIG. 6B shows a graph of the frequency of fragments less than 150 bp in size across the combinatorial jagged end categories according to embodiments of the present invention.

FIG. 6C shows a graph of the frequency of fragments greater than 280 bp in size across the combinatorial jagged end categories according to embodiments of the present invention.

FIGS. 7A and 7B are graphs involving size ratios of different jagged ends according to embodiments of the present invention.

FIG. 8 is graph of the CCCA end motif across different types of ends according to embodiments of the present invention.

FIGS. 9A and 9B illustrate a technique that can combine the jagged end, 5′ end motif, and 3′ end motif to measure the phase of end modalities of cfDNA molecules according to embodiments of the present invention.

FIGS. 10A and 10B illustrate a technique for naming joint end motifs according to embodiments of the present invention.

FIG. 11A is a graph of the correlation of the frequency of the overall 5′ end motif between HCC and healthy subjects.

FIG. 11B is a graph of the correlation of the frequency of the phased end motifs between HCC and healthy subjects according to embodiments of the present invention.

FIG. 11C is a graph of the correlation of the frequency of the joint end motifs between HCC and healthy subjects according to embodiments of the present invention.

FIGS. 12A-12F are graphs of frequency of different jagged end modalities for different nucleic activities according to embodiments of the present invention.

FIG. 13 is a table of the median frequency and relative changes of 5′ A-end, T-end, C-end, and G-end in fragments with 5′ protruding jagged end, 3′ protruding jagged end, and blunt end in WT, DNASE1L3−/−, DNASE1−/−, and DFFB−/− mice according to embodiments of the present invention.

FIGS. 14A-14D are graphs of end motif rankings for DFFB^{-/-} (DFFB knockout [KO]) mice and wildtype (WT) mice according to embodiments of the present invention.

FIG. 15 is a flowchart of an example process of analyzing a biological sample according to embodiments of the present invention.

FIG. 16 is a flowchart of an example process of analyzing a biological sample according to embodiments of the present invention.

FIG. 17 is a flowchart of an example process of analyzing a biological sample according to embodiments of the present invention.

FIGS. 18A-18C show the frequency of different protruding jagged ends for fetal-specific and shared cfDNA fragments according to embodiments of the present invention.

FIG. 18D is a graph of the fetal DNA fractions deduced from fragments having different types of protruding jagged ends according to embodiments of the present invention.

FIG. 19 is a graph of fetal DNA fraction versus different jagged end modalities according to embodiments of the present invention.

FIGS. 20A and 20B are graphs of the fetal DNA fraction deduced from fragments with certain jagged end modalities and sequence end motifs according to embodiments of the present invention.

FIG. 21 is a flowchart of an example process for enriching a biological sample for clinically-relevant DNA according to embodiments of the present invention.

FIG. 22A is a graph of the mRNA expression level of DNASE1 in white blood cell and placenta according to embodiments of the present invention.

FIG. 22B is a graph of the mRNA expression level of DFFB in white blood cell and placenta according to embodiments of the present invention.

FIG. 22C is a graph of the correlation between fetal DNA fraction and the frequency of cfDNA fragments carrying 5′ protruding jagged end according to embodiments of the present invention.

FIG. 22D is a graph of the correlation between fetal DNA fraction and the frequency of cfDNA fragments carrying blunt ends according to embodiments of the present invention.

FIG. 23 is a flowchart of an example process for determining a fraction of clinically-relevant DNA in a biological sample according to embodiments of the present invention.

FIG. 24 illustrates a measurement system according to embodiments of the present invention.

FIG. 25 illustrates a computer system according to embodiments of the present invention.

### TERMS

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissue from different organisms (mother vs. fetus) or to healthy cells vs. tumor cells. “Reference tissues” can correspond to tissues used to determine tissue-specific methylation levels. Multiple samples of a same tissue type from different individuals may be used to determine a tissue-specific methylation level for that tissue type.

An “organ” corresponds to a group of tissues with similar functions. One or more types of tissue can be found in a single organ. Organs may be a part of different organ systems, including the cardiovascular system, digestive system, endocrine system, excretory system, lymphatic system, integumentary system, muscular system, nervous system, reproductive system, respiratory system, and skeletal system.

A “biological sample” refers to any sample that is taken from a subject (e.g., a human, such as a pregnant woman, a person with cancer, or a person suspected of having cancer, an organ transplant recipient or a subject suspected of having a disease process involving an organ (e.g., the heart in myocardial infarction, or the brain in stroke, or the hematopoietic system in anemia) and contains one or more nucleic acid molecule(s) of interest. The biological sample can be a bodily fluid, such as blood, plasma, serum, urine, vaginal fluid, fluid from a hydrocele (e.g. of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g. thyroid, breast), etc. Stool samples can also be used. In various embodiments, the majority of DNA in a biological sample that has been enriched for cell-free DNA (e.g., a plasma sample obtained via a centrifugation protocol) can be cell-free, e.g., greater than 50%, 60%, 70%, 80%, 90%, 95%, or 99% of the DNA can be cell-free. The centrifugation protocol can include, for example, 3,000 g×10 minutes, obtaining the fluid part, and re-centrifuging at for example, 30,000 g for another 10 minutes to remove residual cells.

A “sequence read” refers to a string of nucleotides sequenced from any part or all of a nucleic acid molecule. For example, a sequence read may be a short string of nucleotides (e.g., 20-150) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

An “ending position” or “end position” (or just “end) can refer to the genomic coordinate or genomic identity or nucleotide identity of the outermost base, i.e. at the extremities, of a cell-free DNA molecule, e.g. plasma DNA molecule. The end position can correspond to either end of a DNA molecule. In this manner, if one refers to a start and end of a DNA molecule, both would correspond to an ending position. In practice, one end position is the genomic coordinate or the nucleotide identity of the outermost base on one extremity of a cell-free DNA molecule that is detected or determined by an analytical method, such as but not limited to massively parallel sequencing or next-generation sequencing, single molecule sequencing, double- or single-stranded DNA sequencing library preparation protocols, polymerase chain reaction (PCR), or microarray. Thus, each detectable end may represent the biologically true end or the end is one or more nucleotides inwards or one or more nucleotides extended from the original end of the molecule e.g. 5′ blunting and 3′ filling of overhangs of non-blunt-ended double stranded DNA molecules by the Klenow fragment. The genomic identity or genomic coordinate of the end position could be derived from results of alignment of sequence reads to a human reference genome, e.g., hg19. It could be derived from a catalog of indices or codes that represent the original coordinates of the human genome. It could refer to a position or nucleotide identity on a cell-free DNA molecule that is read by but not limited to target-specific probes, mini-sequencing, DNA amplification.

A “sequence motif” may refer to a short, recurring pattern of bases in DNA fragments (e.g., cell-free DNA fragments). A sequence motif can occur at an end of a fragment, and thus be part of or include an ending sequence. An “end motif” can refer to a sequence motif for an ending sequence that preferentially occurs at ends of DNA fragments, potentially for a particular type of tissue. An end motif may also occur just before or just after ends of a fragment, thereby still corresponding to an ending sequence. A nuclease can have a specific cutting preference for a particular end motif, as well as a second most preferred cutting preference for a second end motif.

The term “length of overhang” between the DNA strands may refer to a value that can be estimated by comparing the jaggedness (e.g., jaggedness index values) of overall plasma DNA or plasma DNA within a certain fragment size range between reference samples (e.g., normal cells) and differentially-regulated nuclease samples (e.g., tumor cells). In some instances, the length of overhang varies based on a specific DNA fragment size range (e.g., 130-160 bp, 200-300 bp) selected for determining a characteristic of the biological sample.

In some embodiments, the length of overhang in the DNA strands is a categorical value that characterize the length of overhang between two DNA strands. For example, a “long” overhang can include an overhang of a DNA strand that has a size of 5 nt, 6 nt, 7 nt, 8 nt, 10 nt, 15 nt, 20 nt, 30 nt, 40 nt, 50 nt, 100 nt, and greater than 100 nt. A “short” overhang can include an overhang of a DNA strand that has a size of 0 nt, 1 nt, 2 nt, 3 nt, 4 nt, 5 nt. Additionally or alternatively, the specified length of overhang in DNA strands can be estimated based on a percentage of molecules that have a size of overhang that exceeds a particular threshold. For instance, a presence of “long” overhang in plasma DNA could be expressed as the percentage of molecules greater than 5 nt, 6 nt, 7 nt, 8 nt, 10 nt, 15 nt, 20 nt, 30 nt, 40 nt, 50 nt, 100 nt, or their combinations.

A “calibration sample” can correspond to a biological sample whose fractional concentration of clinically-relevant DNA (e.g., tissue-specific DNA fraction) is known or determined via a calibration method, e.g., using an allele specific to the tissue, such as in transplantation in a pregnant subject whereby an allele present in the donor's genome but absent in the recipient's genome can be used as a marker for the transplanted organ. As another example, a calibration sample can correspond to a sample from which end motifs can be determined. A calibration sample can be used for both purposes.

A “calibration data point” includes a “calibration value” and a measured or known property of the sample or subject, e.g., age or tissue-specific fraction (e.g., fetal or tumor). The calibration value can be a relative abundance as determined for a calibration sample, for which the property is known. The calibration data point can include the calibration value (e.g., a jagged end value, also called an overhang index) and the known (measured) property. The calibration data points may be defined in a variety of ways, e.g., as discrete points or as a calibration function (also called a calibration curve or calibration surface). The calibration function could be derived from additional mathematical transformation of the calibration data points. The calibration function can be linear or non-linear.

A “site” (also called a “genomic site”) corresponds to a single site, which may be a single base position or a group of correlated base positions, e.g., a CpG site or larger group of correlated base positions. A “locus” may correspond to a region that includes multiple sites. A locus can include just one site, which would make the locus equivalent to a site in that context.

A “separation value” corresponds to a difference or a ratio involving two values, e.g., two fractional contributions or two methylation levels. The separation value could be a simple difference or ratio. As examples, a direct ratio of x/y is a separation value, as well as x/(x+y). The separation value can include other factors, e.g., multiplicative factors. As other examples, a difference or ratio of functions of the values can be used, e.g., a difference or ratio of the natural logarithms (ln) of the two values. A separation value can include a difference and a ratio.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1). The terms “cutoff” and “threshold” refer to predetermined numbers used in an operation. For example, a cutoff size can refer to a size above which fragments are excluded. A threshold value may be a value above or below which a particular classification applies. Either of these terms can be used in either of these contexts.

The term “parameter” as used herein means a numerical value that characterizes a quantitative data set and/or a numerical relationship between quantitative data sets. For example, a ratio (or function of a ratio) between a first amount of a first nucleic acid sequence and a second amount of a second nucleic acid sequence is a parameter.

The terms “cutoff” and “threshold” refer to predetermined numbers used in an operation. For example, a cutoff size can refer to a size above which fragments are excluded. A threshold value may be a value above or below which a particular classification applies. Either of these terms can be used in either of these contexts. A cutoff or threshold may be “a reference value” or derived from a reference value that is representative of a particular classification or discriminates between two or more classifications. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical analyses or simulations of samples. A particular value for a cutoff, threshold, reference, etc. can be determined based on a desired accuracy (e.g., a sensitivity and specificity).

A “pregnancy-associated disorder” includes any disorder characterized by abnormal relative expression levels of genes in maternal and/or fetal tissue or by abnormal clinical characteristics in the mother and/or fetus. These disorders include, but are not limited to, preeclampsia (Kaartokallio et al. Sci Rep. 2015; 5:14107; Medina-Bastidas et al. Int J Mol Sci. 2020; 21:3597), intrauterine growth restriction (Faxen et al. Am J Perinatol. 1998; 15:9-13; Medina-Bastidas et al. Int J Mol Sci. 2020; 21:3597), invasive placentation, pre-term birth (Enquobahrie et al. BMC Pregnancy Childbirth. 2009; 9:56), hemolytic disease of the newborn, placental insufficiency (Kelly et al. Endocrinology. 2017; 158:743-755), hydrops fetalis (Magor et al. Blood. 2015; 125:2405-17), fetal malformation (Slonim et al. Proc Natl Acad Sci USA. 2009; 106:9425-9), HELLP syndrome (Dijk et al. J Clin Invest. 2012; 122:4003-4011), systemic lupus erythematosus (Hong et al. J Exp Med. 2019; 216:1154-1169), and other immunological diseases of the mother.

A “level of pathology” (or level of a disorder or level of condition) can refer to the amount, degree, or severity of pathology associated with an organism. An example is a cellular disorder in expressing a nuclease. Another example of pathology is a rejection of a transplanted organ. Other example pathologies can include autoimmune attack (e.g., lupus nephritis damaging the kidney or multiple sclerosis), inflammatory diseases (e.g., hepatitis), fibrotic processes (e.g., cirrhosis), fatty infiltration (e.g., fatty liver diseases), degenerative processes (e.g., Alzheimer's disease) and ischemic tissue damage (e.g., myocardial infarction or stroke). A heathy state of a subject can be considered a classification of no pathology. The pathology can be cancer.

The term “level of cancer” can refer to whether cancer exists (i.e., presence or absence), a stage of a cancer, a size of tumor, whether there is metastasis, the total tumor burden of the body, the cancer's response to treatment, and/or other measure of a severity of a cancer (e.g., recurrence of cancer). The level of cancer may be a number or other indicia, such as symbols, alphabet letters, and colors. The level may be zero. The level of cancer may also include premalignant or precancerous conditions (states). The level of cancer can be used in various ways. For example, screening can check if cancer is present in someone who is not previously known to have cancer. Assessment can investigate someone who has been diagnosed with cancer to monitor the progress of cancer over time, study the effectiveness of therapies or to determine the prognosis. In one embodiment, the prognosis can be expressed as the chance of a patient dying of cancer, or the chance of the cancer progressing after a specific duration or time, or the chance or extent of cancer metastasizing. Detection can mean ‘screening’ or can mean checking if someone, with suggestive features of cancer (e.g., symptoms or other positive tests), has cancer.

The abbreviation “bp” refers to base pairs. In some instances, “bp” may be used to denote a length of a DNA fragment, even though the DNA fragment may be single stranded and does not include a base pair. In the context of single-stranded DNA, “bp” may be interpreted as providing the length in nucleotides.

The abbreviation “nt” refers to nucleotides. In some instances, “nt” may be used to denote a length of a single-stranded DNA in a base unit. Also, “nt” may be used to denote the relative positions such as upstream or downstream of the locus being analyzed. For a double-stranded DNA, “nt” may still refer to the length of a single strand rather than the total number of nucleotides in the two strands, unless context clearly dictates otherwise. In some contexts concerning technological conceptualization, data presentation, processing and analysis, “nt” and “bp” may be used interchangeably.

The term ‘jagged end’ may refer to sticky ends of DNA, overhangs of DNA, protrusions of strands, or where a double-stranded DNA includes a strand of DNA not hybridized to the other strand of DNA. “Jagged end value” is a measure of the extent of a jagged end. The jagged end value may be proportional to a length of one strand that overhangs a second strand in double-stranded DNA. The jagged end value of a plurality of DNA molecules may include consideration of blunt ends among the DNA molecules.

In some instances, the jagged end value can provide a collective measure of strands that overhangs other strands in a plurality of cell-free DNA molecules. The collective measure of jaggedness can be determined based on an estimated length of overhangs in the plurality of cell-free DNA molecules, e.g., an average, median, or other collective measure of individual measurements of each of the cell-free DNA molecules. In some instances, the collective measure of jaggedness is determined for a particular fragment size range (e.g., 130-160 bp, 200-300 bp).

The term “size ratio” may refer to the amount of cell-free DNA molecules within a particular fragment size range. The size ratio may be proportional to the amount of cell-free DNA molecules within a particular fragment size range normalized by the another amount of cell-free DNA molecules within another particular fragment size range. When the another particular fragment size range is regarding to all size range, the term “size frequency” can be used.

The term “alignment” and related terms may refer to matching a sequence to a reference sequence. The reference sequence may be a reference genome (e.g., human genome) or a sequence of a particular molecule. Such a reference sequence can comprise at least 100 kb, 1 Mb, 10 Mb, 50 Mb, 100 Mb, and more. Such alignment methods cannot be performed manually and are performed by specialized computer software. Alignment may involve lengthy and numerous sequences (e.g., at least 1,000, 10,000, 100,000, 1 million, 10 million, or 100 million sequences). Additionally, alignment may involve variability within the sequence itself or errors within sequence reads. Alignment with such variability or errors therefore may not require an exact match with a reference sequence.

The term “real-time” may refer to computing operations or processes that are completed within a certain time constraint. The time constraint may be 1 minute, 1 hour, 1 day, or 7 days.

The term “subsequence” may refer to a string of bases that is less than the full sequence corresponding to a nucleic acid molecule. For example, a subsequence may include 1, 2, 3, or 4 bases when the full sequence of the nucleic acid molecule includes 5 or more bases. In some embodiments, a subsequence may refer to a string of bases forming a unit where the unit is repeated multiple times in a tandem serial manner. Examples include 3-nt units or subsequences repeated at loci associated with trinucleotide repeat disorders, 1-nt to 6-nt units or subsequences repeated 5 to 50 times as microsatellites, 10-nt to 60-nt units or subsequences repeated 5 to 50 times as minisatellites, or in other genetic elements, such as Alu repeats.

“Clinically-relevant DNA” can refer to DNA of a particular tissue source that is to be measured, e.g., to determine a fractional concentration of such DNA or to classify a phenotype of a sample (e.g., plasma). Examples of clinically-relevant DNA are fetal DNA in maternal plasma or tumor DNA in a patient's plasma or other sample with cell-free DNA. Another example includes the measurement of the amount of graft-associated DNA in the plasma, serum, or urine of a transplant patient. A further example includes the measurement of the fractional concentrations of hematopoietic and nonhematopoietic DNA in the plasma of a subject, or fractional concentration of a liver DNA fragments (or other tissue) in a sample or fractional concentration of brain DNA fragments in cerebrospinal fluid.

The term “concurrent analysis” can refer to using more than one fragmentomic feature. Using only the 5′ end motif of one end of a nucleic acid molecule or using only the jagged end modality (e.g., 5′ end protruding) would not be concurrent analysis. However, using a combination of the jagged end modalities from both ends of a molecule, the combination of one jagged end modality and sequence end motif at one end, or the combination of the jagged end modalities and sequence end motifs at both ends would be part of concurrent analysis.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to +10%. The term “about” can refer to +5%.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

Standard abbreviations may be used, e.g., bp, base pair(s); kb, kilobase(s); pi, picoliter(s); s or sec, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described.

### DETAILED DESCRIPTION

Cell-free DNA (cfDNA) molecules are nonrandomly fragmented, and the fragmentation pattern of cfDNA molecules contains a wealth of molecular information. For example, the characteristic size profile of cfDNA shows a modal frequency at approximately 166 bp, with smaller molecules forming a series of peaks in a 10-bp periodicity (Lo et al. Sci Transl Med. 2010; 2:61ra91). Such size patterns of plasma DNA fragments suggest the presence of both inter- and intra-nucleosome cleavages during the release of DNA molecules into the blood circulation upon cell death and/or apoptosis. Moreover, our group has previously reported that a subset of genomic locations was found to be preferentially cut during the generation of plasma DNA molecules (Chan et al. Proc Natl Acad Sci USA. 2016; 113:E8159-E8168; Jiang et al. Proc Natl Acad Sci USA. 2018; 115:E10925-E10933); such preferred cutting could reflect the tissue of origin of cfDNA (Jiang et al. Proc Natl Acad Sci USA. 2018; 115:E10925-E10933; Sun et al. Proc Natl Acad Sci USA. 2018; 115:E5106-E5114). Furthermore, we have shown that different nucleases are associated with cell-free DNA molecules with characteristic end signatures (i.e., 5′ end motifs and 5′ protruding jagged ends) (Serpas et al. Proc Natl Acad Sci USA. 2019; 116:641-649; Han et al. Am J Hum Genet. 2020; 106:202-214, Ding et al. Clin Chem. 2022; 68:917-926). The 5′ end motif represents the sequence context of the 5′ end of the cfDNA fragment. The 5′ protruding jagged ends represent the 5′ protruding single strand DNA in the cfDNA molecule. Recently, cell-free DNA end signatures showed promising results to serve as liquid biopsy biomarkers (Jiang et al. Cancer Discov. 2020; 10:664-673; Jiang et al. Genome Res. 2020; 30:1144-1153). Jagged ends are also described in US 2020/0056245 A1 and US 2022/0177971 A1, the entire contents of both of which are incorporated herein by reference for all purposes.

In contrast to the widely studied 5′ end motif and 5′ protruding jagged end, the actual 3′ end motif and 3′ protruding jagged end have not been investigated properly mainly due to the artificial modifications occurring during the preparation of the sequencing library. For typical library preparation methods, end repair steps were included. During end repair, the 3′ protruding jagged ends were removed, and the 3′ receded ends were elongated using the opposite 5′ protruding jagged end as a DNA template. Thus, the original 3′ ends were modified, leading to the alteration of nucleotide information proximal to the 3′ end motif as well as the loss of the 3′ protruding jagged end. Moreover, the 3′ protruding jagged ends were removed to form a blunt end. Because of such an end-repair step, the blunt end information deduced from typical library preparation methods was not reliable.

Recently, one group developed a NGS library preparation method, named XACTLY assay, to study jagged ends via an approach based on sequence adapter ligation (Harkins et al. Nucleic Acids Res. 2020; 48:e47). For instance, Harkins Kincaid et al. ligated Y-shape adapters containing a 7 nt-barcode (i.e., Unique End Identifier (UEI) that denotes a discrete terminus type and length) directly to the original DNA templet without end repair steps (Harkins Kincaid et al. Nucleic Acids Res. 2020; 48:e47). The ligated product was subjected to short-read sequencing (i.e., Illumina sequencing platforms). The native ends of the DNA molecules were supposedly able to be deduced according to the sequence information of 7 nt-barcodes linked to reads. However, as reported in Harkins et al.'s study, 5′ UEIs (i.e., Illumina P5 adapter) had much less fidelity than the 3′ UEIs (i.e., Illumina P7 adapter) (Harkins Kincaid et al. Nucleic Acids Res. 2020; 48:e47). Such an inaccuracy associated with the P5 adapter was because the first ligation of P5 adapter to template DNA would occur regardless of whether the protruding end of the adapter was properly matched with the protruding end on template DNA, whereas the P7 adapter ligation could occur only when the first ligation event was correct. In other words, the P5 adapter ligation would happen, even when the gaps (i.e., areas where one strand has no complementary nucleotide(s) on the other strand) and flaps (i.e., areas where nucleotides of one adapter are not hybridized to a strand) exist in the hybridization area between the template DNA and adapter sequence. In the sequencing library prepared by XACTLY assay, a double-stranded DNA will be denatured into two single-stranded DNA molecules. The 5′ and 3′ ends of such a single-stranded DNA molecule were tagged by P5 and P7, respectively. Therefore, there are intrinsic limitations present in XACTLY assay:
1. At least one end cannot be analyzed in an accurate manner using XACTLY assay.
2. One cannot simultaneously analyze both strands of a DNA molecule effectively.
3. The actual lengths of DNA molecules cannot be measured.

In the present disclosure, we developed new approaches to concurrently detect native fragmentomic features of a cfDNA molecule in high fidelity, including fragment sizes, end motifs, and jagged ends. In one embodiment, using a DNA ligase, a double-stranded cfDNA fragment can be properly ligated with a pair of hairpin adapters, depending on the end modalities of such a double-stranded cfDNA molecule, forming a circularized DNA molecule. Such hairpin adapters contain molecular barcodes and carry jagged ends with various lengths or blunt end. The different cfDNA fragments would be ligated with hairpin adapters containing distinct molecular barcodes which correspond to the jagged end lengths (e.g., 1-50 nt) and jagged types (blunt ends, 5′ protruding jagged ends, 3′ protruding jagged ends, and combinations thereof). The ligation product can be treated with enzymes to remove incomplete circular DNA molecules, thus enriching the desired circular DNA molecules generated by the hairpin adapters mediated DNA ligation (i.e., a negative selection step).

The product enriched for circular DNA molecules may further undergo the direct enrichment of circular DNA molecules such as single-molecule real-time sequencing (e.g., Pacific Biosciences) and rolling cycle amplification (i.e., a positive selection step), to minimize the influence of inaccurate ligations. As only a complete circular DNA molecule could be sequenced multiple times, generating subreads during single-molecule real-time sequencing, the selection of readout with three or more subreads allows for ruling out the incomplete circular DNA molecules. Similarly, only a complete circular DNA molecule could be amplified via rolling circle amplification. In some embodiments, the enzyme includes but is not limited to exonuclease I, exonuclease II, exonuclease III, exonuclease IV, exonuclease V, exonuclease VI, exonuclease VII, or exonuclease VIII. In yet another embodiment, the negative and positive selection steps can be done alone or in combination. After sequencing, one can deduce the native jagged ends of a single cfDNA fragment through analyzing the barcode sequences. Once the jagged ends for each terminus of a cfDNA molecule are determined, the entire fragmentomic features from a cfDNA molecule can be therefore analyzed accurately at a 1-nt resolution, including but not limited to, 5′ protruding jagged end, 3′ protruding jagged end, 5′ receded jagged end, 3′ receded jagged end, end motif of the protruding jagged end, end motif of the receded jagged ends, genomic coordinates of fragment ends, fragment sizes, methylation associated cfDNA fragmentomic features, as well as combinations thereof. In one embodiment, the length difference between the Watson and Crick stands can be used as another type of fragmentomic feature.

In some embodiments, the end motif may be defined by one or more nucleotides across positions at or near an end of a molecule. One molecule can have 4 ends. The end motif may be defined by one or more nucleotides in a reference genome surrounding the genomic locus to which the end of a fragment is aligned. In other embodiments, the jagged end may be defined by the protruding single-strand DNA at the end of the DNA fragment. The jagged end can be separated into different groups according to the length and strand of the protruding single-strand DNA. In yet other embodiments, different fragmentomic features from one DNA fragment can be combined. In one embodiment, the combined fragmentomic features can be used for the detection or monitoring of cancer or other diseases. In another embodiment, combined fragmentomic features can be used for noninvasive prenatal testing. End motifs are described in US 2021/0238668 A1, the entire contents of which are incorporated herein by reference.

### I. Principle of Concurrent Analysis of Single-Molecule End Modalities

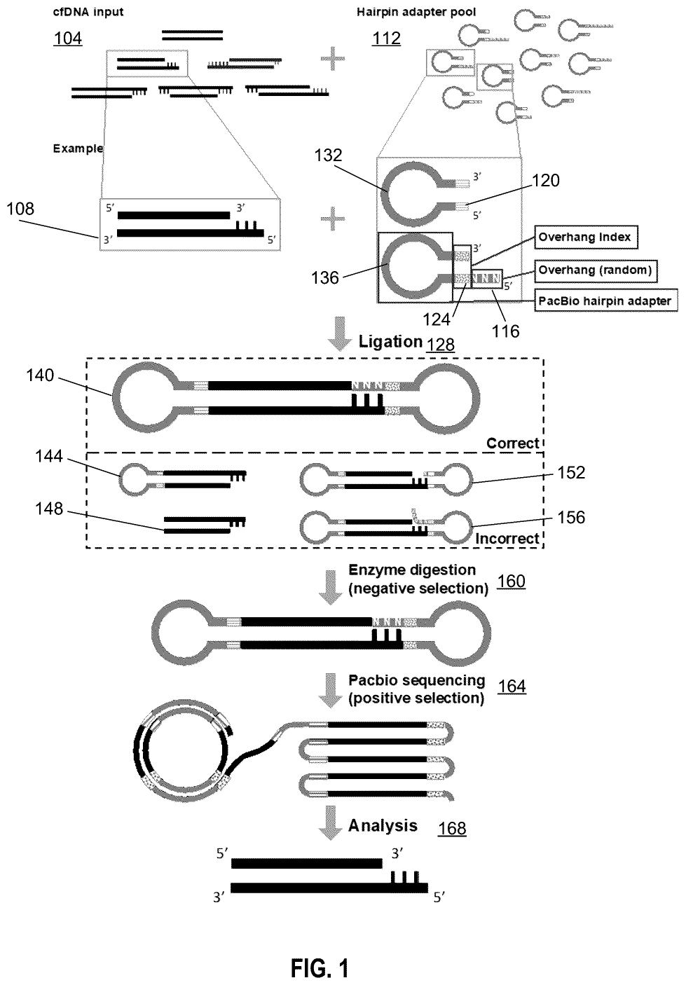

FIG. 1 shows a schematic overview of the concurrent analysis of single-molecule end modalities on a single-molecule real-time sequencing platform (e.g., Pacific Biosciences (PacBio) platform). Stage 104 shows different cfDNAmolecules, which contain different jagged ends or blunt ends. Example fragment 108 has a blunt end at the left side and a 3-nt 5′ protruding jagged end at the right side.

Stage 112 shows different hairpin adapters in the hairpin adapter pool. The hairpin adapter pool contains adapters with blunt ends and adapters with jagged ends (also referred to as overhangs). Each hairpin adapter with a jagged end has a protruding single-strand end of various lengths (indicated by a number of “N” in overhang 116). A barcode sequence synthesized together with the hairpin adapter, which is compatible for PacBio sequencing platform, can be used to indicate the jagged end type (e.g., 5′ or 3′ protruding end) and jagged end length (denoted by the rectangles 120 and 124 filled with different patterns).

At stage 128, the cfDNA molecules are ligated with hairpin adapters. Fragment 108 is ligated with hairpin adapter 132 on its blunt end (left side). Fragment 108 is ligated with hairpin adapter 136 on its 3-nt 5′ protruding jagged end (right side). Proper ligation results in molecule 140.

Other molecules may result from ligation. Molecule 144 represents a fragment that has a hairpin adapter ligated to only one end. Molecule 148 represents a fragment that has no hairpin adapters. Molecule 152 has a hairpin adapter ligated correctly to the blunt end. However, molecule 152 has a hairpin adapter ligated incorrectly to the 5′ protruding end, with a gap between the cfDNA fragment and the hairpin adapter. Molecule 156 has a hairpin adapter ligated correctly to the blunt end. However, molecule 156 has a hairpin adapter ligated incorrectly to the 5′ protruding end, with the hairpin adapter creating a flap, where nucleotides of the hairpin adapter are not hybridized to the original cfDNA fragment.

At stage 160, the adapter-ligated molecules may be treated with an enzyme/enzymes that can digest incomplete circular DNA molecules (e.g., molecule 152 and molecule 156). The enzyme digestion of incomplete adapter-ligated molecules may be referred to as negative selection because the incorrectly ligated molecules are selected and removed.

At stage 164, the enzyme-treated ligation product can be sequenced on the PacBio platform. Only when cfDNA fragments with both ends are properly ligated with hairpin adapters that correspond to the native jagged ends to form complete circular DNA (e.g., through rolling circle amplification), such a circular DNA product can be sequenced to generate multiple subreads for each strand. The amplification and/or sequencing of only complete adapter-ligated molecules may be referred to as positive selection because the correctly ligated molecules are selected and further analyzed.

At stage 168, the sequences are analyzed. After sequencing, one can read the barcode sequence information at both ends to deduce the presence of jagged ends and/or blunt ends, and the type and length of a jagged end if present. Based on deduced ends, we can further detect the 5′ end motif, 3′ end motif, and/or the size of each strand of a cfDNA fragment. Native fragmentomic features of the original cfDNA molecules may be assessed.

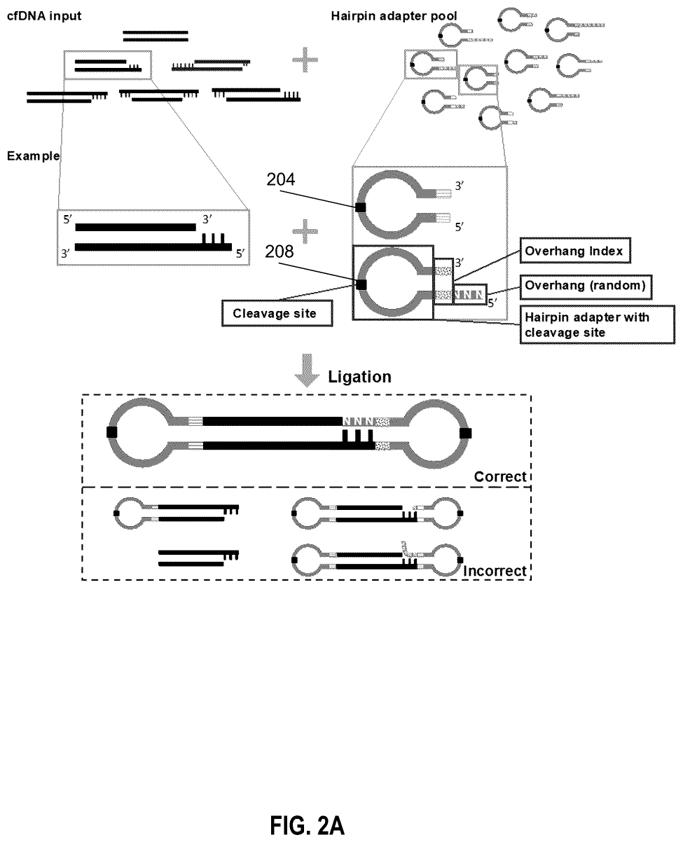

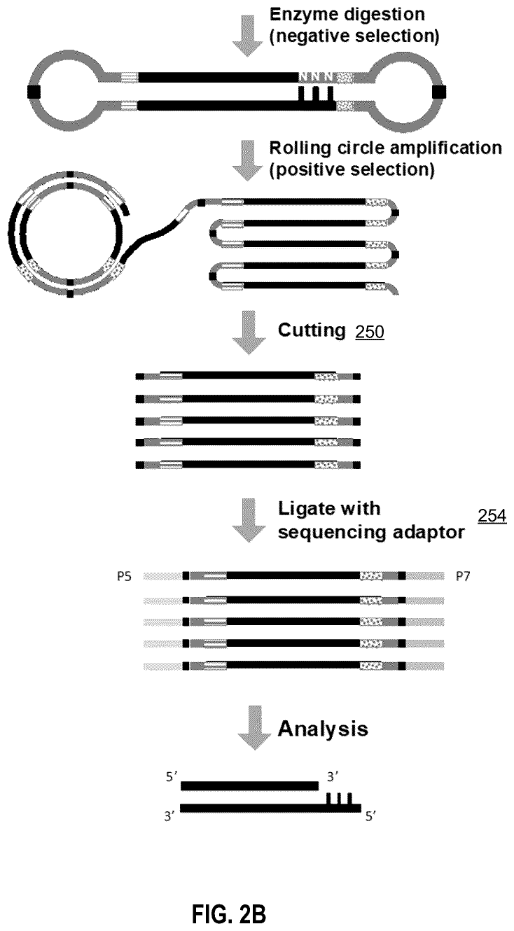

FIGS. 2A and 2B show a schematic overview of the concurrent analysis of single-molecule end modalities on a next generation sequencing (NGS) platform (e.g., Illumina platform). The circular cfDNA molecules can be prepared according to the embodiments in this disclosure with modified hairpin adapters. Stage 104 may be repeated in FIG. 2A. Stage 112 may include modified hairpin adapters, which contain a cleavage site for a restriction enzyme. For example, cleavage sites 204 and 208 may be included in the hairpin adapters.

In FIG. 2B, similar to stage 128, DNA fragments are ligated with hairpin adapters, and similar to stage 160, the adapter-ligated molecules would be treated with an enzyme/enzymes which can digest incomplete circular DNA molecules (i.e., negative selection). Similar to stage 160, the enzyme-treated ligation product would be amplified through rolling circle amplification (i.e., positive selection). Only cfDNA fragments with both ends properly ligated with hairpin adapters that correspond to the native jagged ends/blunt ends would be amplified.

At stage 250, the rolling-amplified product is treated with the specified restriction enzyme to cut at the cleavage site in the hairpin adapter. Hence, large DNA molecules generated via rolling PCR are cut into small DNA molecules, which are suitable for Illumina sequencing or other similar sequencing.

At stage 254, sequencing adapters are ligated onto the cleaved small DNA molecules. The sequencing adapters are configured for Illumina sequencing.

Analysis in FIG. 2B may be similar to stage 168 of FIG. 1.

### A. Example Positive Selection Method

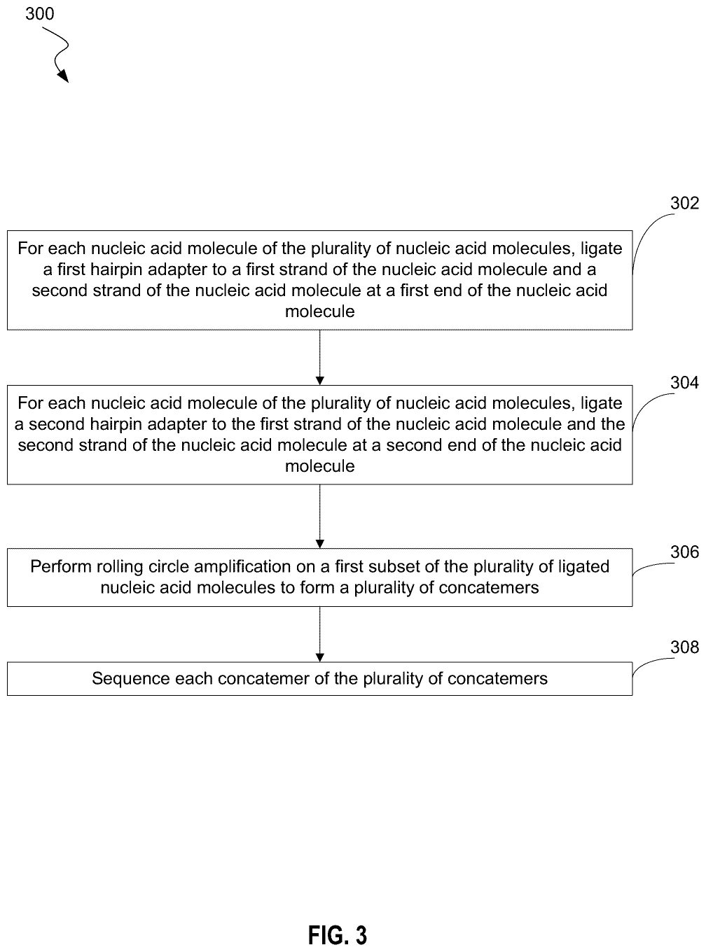

FIG. 3 is a flowchart of an example process 300 of analyzing a biological sample. Process 300 may determine whether a jagged end exists at both ends of a cfDNA molecule, whether the 5′ or 3′ end is protruding, lengths of the overhang, and/or the sequence of the overhang. A strand that overhangs another strand may be understood to be protruding. In some implementations, one or more process blocks of FIG. 3 may be performed by a system, including system 2400. The biological sample may include a plurality of nucleic acid molecules. The nucleic acid molecules may be cell-free and double-stranded with a first strand and a second strand.

At block 302, for each nucleic acid molecule of the plurality of nucleic acid molecules, a first hairpin adapter is ligated to a first strand of the nucleic acid molecule and a second strand of the nucleic acid molecule at a first end of the nucleic acid molecule. The first hairpin adapter may include a first sequence identifier. The first sequence identifier may identify a first length of zero or more nucleotides at a first terminus of the first hairpin adapter having no complementary portion at a second terminus of the first hairpin adapter. The length of nucleotides with no complementary portion in the hairpin corresponds to the length of a jagged end of the nucleic acid molecule. Zero nucleotides signify a blunt end and where the hairpin adapter ends are complementary. For example, the first sequence identifier may encode that the length is 0, 1, 2, 3, 4, 5, 6, 7, 8, 9, or 10 nucleotides. The first sequence identifier may encode whether the 3′ strand or the 5′ strand of the nucleic acid molecule to which the first hairpin ligates is overhanging the other. In some embodiments, the first sequence identifier may encode a subsequence of the zero or more nucleotides at the first terminus of the first hairpin adapter having no complementary portion at a second terminus of the first hairpin adapter.

The first hairpin adapter may include hairpin adapters 132 and 136 in FIG. 1. The first sequence identifier may be the nucleotides represented by rectangles 120 and 124. The length of zero or more nucleotides at the first terminus of the first hairpin adapter having no complementary portion at a second terminus of the first hairpin adapter may include overhang 116.

At block 304, for each nucleic acid molecule of the plurality of nucleic acid molecules, a second hairpin adapter is ligated to the first strand and the second strand at a second end of the nucleic acid molecule. The second hairpin adapter may include a second sequence identifier. The second sequence identifier may identify a second length of zero or more nucleotides at a first terminus of the second hairpin adapter having no complementary portion at a second terminus of the second hairpin adapter. The second sequence identifier may have similar properties as the first sequence identifier. The first sequence identifier and the second sequence identifier may use similar encodings. Certain predetermined subsequences in the sequence identifiers may correspond to different numbers. With four nucleotides (A, T, G, C), the length may be represented in numerical base 4. A plurality of ligated nucleic acid molecules is generated after ligating.

In some examples, negative selection may be performed. Exonucleases may be added to the plurality of ligated nucleic acid molecules after ligating the plurality of first hairpin adapters and the plurality of second hairpin adapters to remove an incorrectly-ligated subset of the plurality of ligated nucleic acid molecules. For each nucleic acid molecule of the incorrectly-ligated subset, either the respective nucleic acid molecule is not completely hybridized to the respective first hairpin adapter or the respective second hairpin adapter (e.g., a “gap” exists), or the respective first hairpin adapter or the respective second hairpin adapter is not completely hybridized to the respective nucleic acid molecule (e.g., a “flap” exists). Negative selection may be similar to stage 160 of FIG. 1, where molecules 144, 148, 152, and 156 are removed.

At block 306, rolling circle amplification may be performed on a first subset of the plurality of ligated nucleic acid molecules to form a plurality of concatemers. The first subset may not include any of the same nucleic acid molecules as the incorrectly-ligated subset. Each nucleic acid molecule of the first subset may be ligated to a respective first hairpin adapter of a plurality of first hairpin adapters and a respective second hairpin adapter of a plurality of second hairpin adapters. Each nucleic acid molecule of the first subset may be correctly ligated to the hairpin adapters, without gaps or flaps, similar to molecule 140 in FIG. 1. Each nucleotide of a strand of the nucleic acid molecule of the first set may by hybridized to a complementary nucleotide on the other strand.

Each nucleic acid molecule of a first portion of the first subset may have the respective first strand overhanging the respective second strand at the respective first end. The first strand may be the 5′ strand or the 3′ strand at the first end. In some examples, each nucleic acid molecule of a second portion of the first subset may have the respective first strand even with the respective second strand at the respective first end. In some examples, each nucleic acid molecule of a second portion of the first subset has the respective second strand overhanging the respective first strand at the respective first end. The respective first strand may be the 5′ strand. The respective second strand may be the 3′ strand.

The first subset may include portions corresponding to the different combinatorial jagged end properties: DNA molecules containing the 5′ protruding jagged end and 3′ protruding jagged end (5-3); 5′ protruding jagged end and 5′ protruding jagged end (5-5); 3′ protruding jagged end and 3′ protruding jagged end (3-3); 5′ protruding jagged end and blunt end (5-B); 3′ protruding jagged end and blunt end (3-B); and blunt end and blunt end (B-B).

At block 308, each concatemer of the plurality of concatemers is sequenced to identify the respective first sequence identifier and the respective second sequence identifier. The first sequence identifier and the second sequence identifier may each include a subsequence of nucleotides indicating that consecutive nucleotides are part of the identifier. Sequencing may be through single-molecule, real time sequencing, next generation sequencing, or any suitable sequencing technique. Sequencing may occur simultaneously with the performing of the rolling circle amplification.

Lengths of overhangs present at the first ends of nucleic acid molecules of the first subset of the plurality of ligated nucleic acid molecules may be determined using the first sequence identifiers. The first sequence identifier may include a subsequence corresponding to the length of the overhang. Additionally, the first sequence identifier may include a subsequence that indicates whether the overhang is on the present strand or the complementary strand.

Lengths of overhangs present at the second ends of nucleic acid molecules of the first subset of the plurality of ligated nucleic acid molecules are determined using the second sequence identifiers. The second sequence identifiers may be used in a similar manner as the first sequence identifiers.

In some examples, the first sequence end motifs of overhangs present at the first ends of nucleic acid molecules of the first subset of the plurality of ligated nucleic acid molecules may be determined using the sequences of the first sequence identifiers. The first sequence identifier may indicate which strand at an end is protruding, and the appropriate subsequence can be associated with the overhang. Additionally, the first sequence identifier indicates the length of the overhang, so the entire sequence of the overhang may be determined. In some embodiments, the entire sequence of the overhang may not be determined, and instead an end motif (of 2, 3, 4, 5, 6, 7, 8, 9, or 10 nucleotides) may be determined. In some examples, the second sequence end motifs of overhangs present at the second ends of nucleic acid molecules of the first subset of the plurality of ligated nucleic acid molecules may be determined using the sequences of the second sequence identifiers.

In some examples, whether a 5′ strand or a 3′ strand overhangs the other may be determined for each nucleic acid molecule having an overhang at the first end of the first subset using the sequence of the respective first identifier. In some examples, whether a 5′ strand or a 3′ strand overhangs the other may be determined for each nucleic acid molecule having an overhang at the second end of the first subset using the sequence of the respective second identifier.

In some examples, each first hairpin adapter of the plurality of first hairpin adapters may include a first cleavage site. Each second hairpin adapter of the plurality of second hairpin adapters may include a second cleavage site. The process may include cleaving each concatemer of the plurality of concatemers at a respective first cleavage site and at a respective second cleavage site.

Process 300 may be used to determine lengths or end motifs in other processes disclosed herein. In some examples, each nucleic acid molecule of the plurality of molecules has a size greater than a first cutoff size. In some examples, each nucleic acid molecule of the plurality of molecules has a size less than a second cutoff size. The first cutoff size and the second cutoff size may independently be 90, 100, 110, 120, 130, 140, 150, 160, 170, 180, 190, 200, 210, 220, 230, 240, 250, 260, 270, 280, 290, 300, 310, 320, 330, 340, or 350. The size of each nucleic acid molecule may be determined by aligning subsequences corresponding to the ends of the respective nucleic acid molecule with a reference genome.

The condition may be cancer (for example, but not limited to, HCC and colorectal cancer [CRC]), an autoimmune disease (e.g., systemic lupus erythematosus), a pregnancy-associated disorder, or any condition described herein. The reference value may be determined from one or more subjects having a certain level of the condition or one or more healthy subjects.

In some examples, the level of the condition is not determined. Instead, a fractional concentration of clinically-relevant DNA may be determined using the comparison. The reference value may be determined from one or more subjects having a known fractional concentration of clinically-relevant DNA. The reference value may be a calibration value determined using a calibration sample.

In some examples, reads corresponding to the plurality of nucleic acid molecules can be enriched for clinically-relevant DNA. For example, the biological sample may be obtained from a female subject pregnant with a fetus. The method may further include selecting reads corresponding to a subset of nucleic acid molecules having the 5′ strand or the 3′ strand overhanging the other end. The method may include analyzing the subset of nucleic acid molecules for a characteristic of the fetus. For example, the characteristic may be the presence of an aberration (e.g., mutation, aneuploidy) in the fetal genome. As another example, the reads may be enriched for the maternal sample by selecting reads having blunt ends at one end. Other clinically-relevant DNA can be enriched by analyzing the concentration of such DNA among different jagged end modalities. The jagged end modalities with higher concentrations of the clinically-relevant DNA can be selected to result in an enriched data set. End modalities may include end modalities at the two ends of any given fragment.

Process 300 may include additional implementations, such as any single implementation or any combination of implementations described herein and/or in connection with one or more other processes described elsewhere herein.

Although FIG. 3 shows example blocks of process 300, in some implementations, process 300 may include additional blocks, fewer blocks, different blocks, or differently arranged blocks than those depicted in FIG. 3. Additionally, or alternatively, two or more of the blocks of process 300 may be performed in parallel.

### B. Example Negative Selection Method

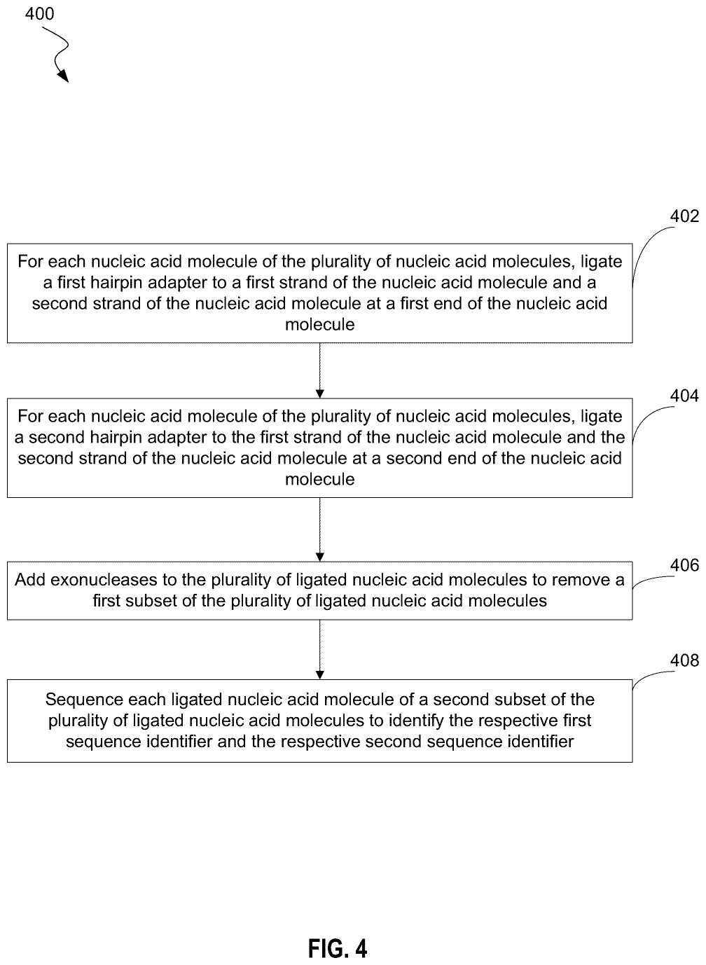

FIG. 4 is a flowchart of an example process 400 of analyzing a biological sample. Process 400 may determine whether a jagged end exists at both ends of a cfDNA molecule, whether the 5′ or 3′ end is protruding, lengths of the overhang, and/or the sequence of the overhang. A strand that overhangs another strand may be understood to be protruding. In some implementations, one or more process blocks of FIG. 4 may be performed by a system 2400.

At block 402, for each nucleic acid molecule of the plurality of nucleic acid molecules, a first hairpin adapter is ligated to a first strand of the nucleic acid molecule and a second strand of the nucleic acid molecule at a first end of the nucleic acid molecule. Block 402 may be performed in the same manner as block 302.

At block 404, for each nucleic acid molecule of the plurality of nucleic acid molecules, a second hairpin adapter is ligated to the first strand and the second strand at a second end of the nucleic acid molecule. Block 404 may be performed in the same manner as block 304.

At block 406, exonucleases are added to the plurality of ligated nucleic acid molecules to remove a first subset of the plurality of ligated nucleic acid molecules. For each nucleic acid molecule of the first subset, either the respective nucleic acid molecule is not completely hybridized to the respective first hairpin adapter or the respective second hairpin adapter, or the respective first hairpin adapter or the respective second hairpin adapter is not completely hybridized to the respective nucleic acid molecule.

At block 408, each ligated nucleic acid molecule of a second subset of the plurality of ligated nucleic acid molecules may be sequenced to identify the respective first sequence identifier and the respective second sequence identifier. The second subset is the ligated nucleic acid molecules that remain in the biological sample after removing the first subset. Sequencing may be performed by next generation sequencing, single-molecule real time sequencing, or any sequencing technique described herein.

Lengths of overhangs present at the first ends of nucleic acid molecules of the second subset of the plurality of ligated nucleic acid molecules may be determined using the first sequence identifiers. The first sequence identifier may include a subsequence corresponding to the length of the overhang. Additionally, the first sequence identifier may include a subsequence that indicates whether the overhang is on the present strand or the complementary strand.

Lengths of overhangs present at the second ends of nucleic acid molecules of the second subset of the plurality of ligated nucleic acid molecules are determined using the second sequence identifiers. The second sequence identifiers may be used in a similar manner as the first sequence identifiers.

In some examples, the first sequence end motifs of overhangs present at the first ends of nucleic acid molecules of the second subset of the plurality of ligated nucleic acid molecules may be determined using the sequences of the first sequence identifiers. The first sequence identifier may indicate which strand at an end is protruding, and the appropriate subsequence can be associated with the overhang. Additionally, the first sequence identifier indicates the length of the overhang, so the entire sequence of the overhang may be determined. In some embodiments, the entire sequence of the overhang may not be determined, and instead an end motif (of 2, 3, 4, 5, 6, 7, 8, 9, or 10 nucleotides) may be determined. In some examples, the second sequence end motifs of overhangs present at the second ends of nucleic acid molecules of the second subset of the plurality of ligated nucleic acid molecules may be determined using the sequences of the second sequence identifiers.

In some examples, whether a 5′ strand or a 3′ strand overhangs the other may be determined for each nucleic acid molecule having an overhang at the first end of the second subset using the sequence of the respective first identifier. In some examples, whether a 5′ strand or a 3′ strand overhangs the other may be determined for each nucleic acid molecule having an overhang at the second end of the second subset using the sequence of the respective second identifier.

In some examples, each first hairpin adapter of the plurality of first hairpin adapters may include a first cleavage site. Each second hairpin adapter of the plurality of second hairpin adapters may include a second cleavage site. The process may include cleaving each concatemer of the plurality of concatemers at a respective first cleavage site and at a respective second cleavage site.

In some examples, the sample may be enriched for clinically-relevant DNA, as explained with process 300.

Process 400 may include additional implementations, such as any single implementation or any combination of implementations described herein and/or in connection with one or more other processes (including process 300) described elsewhere herein.

Although FIG. 4 shows example blocks of process 400, in some implementations, process 400 may include additional blocks, fewer blocks, different blocks, or differently arranged blocks than those depicted in FIG. 4. Additionally, or alternatively, two or more of the blocks of process 400 may be performed in parallel.

### II. Condition Analysis and Detection

Various conditions can be analyzed and detected. Cancer and nuclease activity deficiencies are examples of a condition that can be analyzed and detected using jagged end modalities and/or sequence end motifs. Other conditions, including conditions characterized by an abnormal nuclease activity, can also be analyzed and detected.

### A. Cancer

For illustration purposes, the sequencing libraries of plasma DNA samples from one healthy subject and one hepatocellular carcinoma (HCC) patient were prepared, respectively. These sequencing libraries were sequenced using the PacBio sequencing platform, obtaining 59,133 and 227,198 circular consensus sequencing (CCS) reads, respectively. Hairpin adapters with blunt ends, 5′ protruding jagged ends (length ranging from 1 to 10 nt), and 3′ protruding jagged ends (length ranging from 1 to 10 nt) were used.

1. Detection with Jagged End Deduced from Concurrent Analysis of Single-Molecule End Modalities

It was reported that the presence of a 5′ protruding jagged end is higher in HCC plasma DNA samples compared with healthy controls (Jiang et al., Genome Res. 2020; 30:1144-1153). In embodiments, 5′ protruding jagged end, 3′ protruding jagged end, and blunt end can be deduced from the concurrent analysis of single-molecule end modalities in a more precise, accurate, and comprehensive manner, potentially improving the diagnostic power.

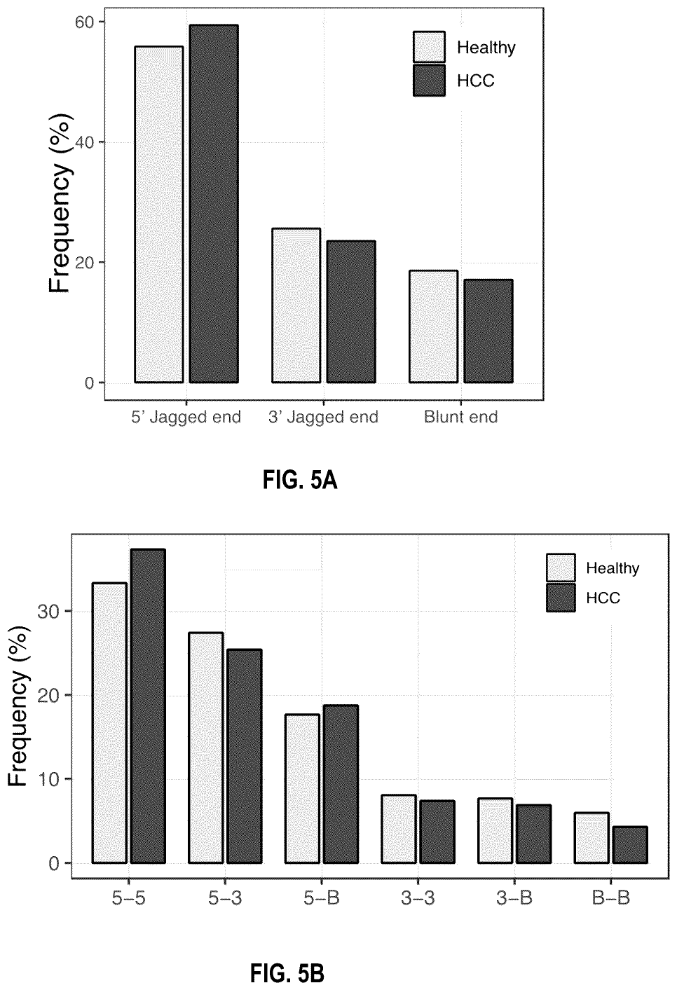

FIG. 5A shows the frequency of the 5′ protruding jagged end, 3′ protruding jagged end, and blunt end in the HCC and healthy subject. The y-axis shows the frequency. The x-axis shows the type of jagged end or blunt end. Two different bars show healthy subject versus a subject with HCC. Two-sided jagged ends from one cfDNA fragment were analyzed separately. The frequency is based on the total number of ends (two ends per molecule) rather than the total number of molecules.

As shown in FIG. 5A, the frequency of the 5′ protruding jagged end was higher in the HCC case (59.40% vs. 55.84%) compared with the healthy subject. A slight decrease was observed in HCC cases in the 3′ protruding jagged end (23.51% vs. 25.57%) and blunt end (17.09% vs. 18.59%).

FIG. 5B shows the frequency of molecules across combinatorial jagged end categories. The y-axis shows the frequency as a percent. The x-axis shows the different combinatorial jagged end properties for both ends of each molecule: a population of DNA molecules containing the 5′ protruding jagged end and 3′ protruding jagged end (5-3); 5′ protruding jagged end and 5′ protruding jagged end (5-5); 3′ protruding jagged end and 3′ protruding jagged end (3-3); 5′ protruding jagged end and blunt end (5-B); 3′ protruding jagged end and blunt end (3-B); and blunt end and blunt end (B-B). Two different bars show the HCC subject and the healthy subject. Two-sided jagged ends from one cfDNA fragment were analyzed concurrently.

As shown in FIG. 5B, the HCC case showed a higher amount of cfDNA fragments that belong to the category 5-5 (37.32% vs. 33.32%) and 5-B (18.75% vs. 17.65%) group, but a lower amount of cfDNA fragments that belong to the category 5-3 (25.41% vs. 27.39%) and B-B group (4.28% vs. 5.94%), compared with the healthy sample. The accumulated difference across these categories between the HCC and healthy subjects is higher when we concurrently analyze both ends (10.21%) compared with a single end (7.12%). These results indicate that the concurrent analysis of jagged end modalities from both sides of a cfDNA fragment can provide more detailed information, which was not available in the previously published technologies and enhance the diagnosis power.

2. Detection with Fragment Size Deduced from the Concurrent Analysis of Single-Molecule End Modalities

In one embodiment, jagged end and fragment size deduced from the concurrent analysis of single-molecule end modalities can be analyzed along with the jagged end category.

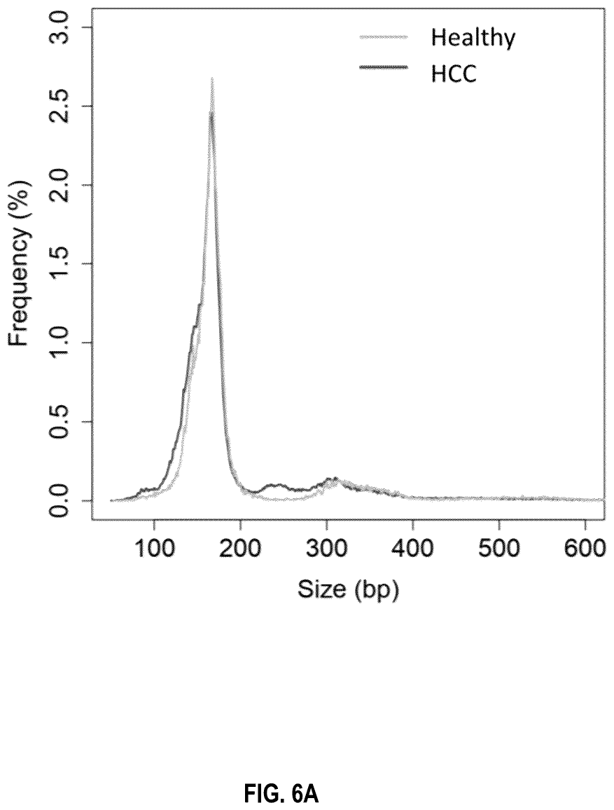

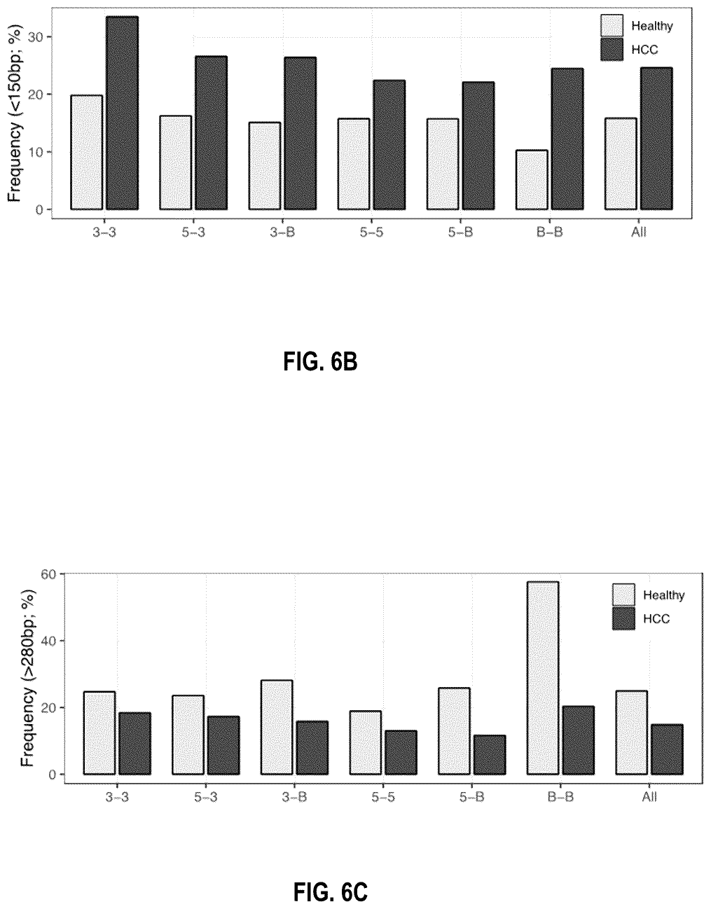

FIG. 6A shows a graph of the overall size distribution of plasma DNA samples from a healthy subject and an HCC subject. The y-axis shows the frequency in percent. The x-axis shows the size in bp. The fragment size is slightly shorter in the HCC case compared with the healthy subject.

FIG. 6B shows a graph of the frequency of fragments less than 150 bp in size across the combinatorial jagged end categories. The y-axis is the frequency in percent in that category having sizes less than 150 bp over molecules of all sizes in that category. The x-axis lists the combinatorial jagged end categories: 5′ protruding jagged end and 3′ protruding jagged end (5-3); 5′ protruding jagged end and 5′ protruding jagged end (5-5); 3′ protruding jagged end and 3′ protruding jagged end (3-3); 5′ protruding jagged end and blunt end (5-B); 3′ protruding jagged end and blunt end (3-B); and blunt end and blunt end (B-B). The x-axis also lists all cfDNA fragments. Two-sided ends from one cfDNA fragment were analyzed concurrently.

FIG. 6C shows a graph of the frequency of fragments greater than 280 bp in size across the combinatorial jagged end categories. The y-axis is the frequency in percent. The x-axis lists the combinatorial jagged end categories and all cfDNA fragments.

As shown in the “All” cfDNA fragments category of FIG. 6B, the frequency of short cfDNA fragments (<150 bp) is higher in the HCC case (24.58% vs. 15.82%). In contrast, as shown in the “All” category of FIG. 6C, the frequency of long cfDNA fragments (>280 bp) is lower in the HCC case compared with the healthy subject (14.81% vs. 24.94%).

We then separated cfDNA fragments into different groups according to the jagged end types of both ends. As shown in FIG. 6B, compared with all cfDNA fragments (24.58% vs. 15.82%), the populations of cfDNA fragments with the blunt end at both ends (B-B; 24.44% vs. 10.27%), and cfDNA fragments with 3′ protruding jagged end at both ends (3-3; 33.45% vs. 19.78%), and cfDNA fragments with 3′ protruding jagged end and blunt end (3-B; 26.38% vs. 15.09%) showed a greater difference in the frequency of short cfDNA fragments between the HCC case and the healthy case.

As shown in FIG. 6C, the populations of cfDNA fragments with the blunt end at both ends (B-B; 20.29% vs. 57.60%), and cfDNA fragments with 5′ protruding jagged end and a blunt end (5-B; 11.61% vs. 25.83%), and cfDNA fragments with 3′ protruding jagged end and blunt end (3-B; 15.78% vs. 28.11%) showed a greater difference in the frequency of long cfDNA fragments between the HCC case and the healthy case compare to all cfDNA fragments (14.81% vs. 24.94%).

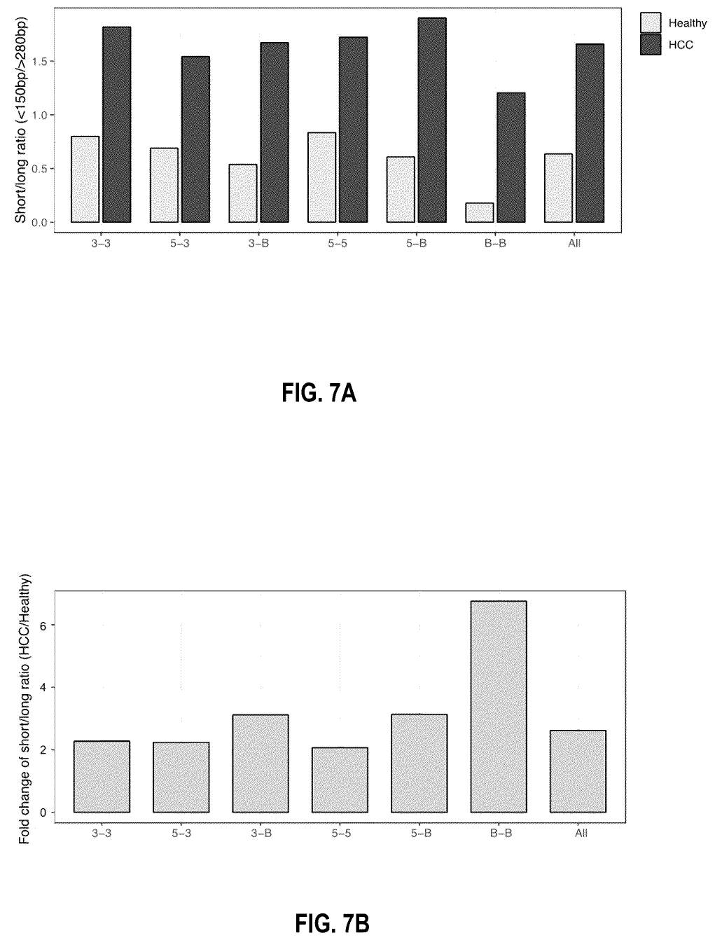

FIG. 7A is a graph of the ratio of short to long fragments for different types of jagged ends. The y-axis is the ratio of short (<150 bp) fragments to long (>280 bp) fragments. The x-axis is the combinatorial jagged end categories and all cfDNA fragments. The different bars show the HCC case and the healthy case. Two-sided ends from one cfDNA fragment were analyzed concurrently.

FIG. 7B is a graph of the fold change of the short/long ratio for the HCC case to healthy case. The y-axis is the fold change, which is calculated from the short/long ratio of the HCC case divided by the short/long ratio of the healthy case. The x-axis is the combinatorial jagged end categories and all cfDNA fragments.

The difference in short/long ratio (i.e., the amount of fragment <150 bp/the amount of fragment >280 bp) between the HCC case and the healthy case was increased in cfDNA fragments with the blunt end at both ends (B-B; 1.20 vs. 0.18; fold change: 6.75), and cfDNA fragments with 5′ protruding jagged end and blunt end (5-B; 1.90 vs. 0.61; fold change: 3.13), as well as cfDNA fragments with 3′ protruding jagged end and blunt end (3-B; 1.67 vs. 0.53; fold change: 3.11), when compared with all fragments (All; 1.65 vs. 0.63; fold change: 2.61). FIGS. 7A and 7B show that certain types of jagged ends or certain combinations of jagged ends may be as effective or more effective in distinguishing between a healthy case and an HCC case as using fragments without consideration for their jagged end type.

3. Detection with End Motif Deduced from the Concurrent Analysis of Single-Molecule End Modalities

The presence of 5′ CCCA end motif was reported to decrease in plasma DNA samples of patients with HCC compared with healthy subjects (Jiang et al. Cancer Discov. 2020; 10:664-673). In embodiments, the 5′ CCCA end motif can be calculated in the 5′ protruding jagged end, 3′ protruding jagged end, and blunt end, separately.

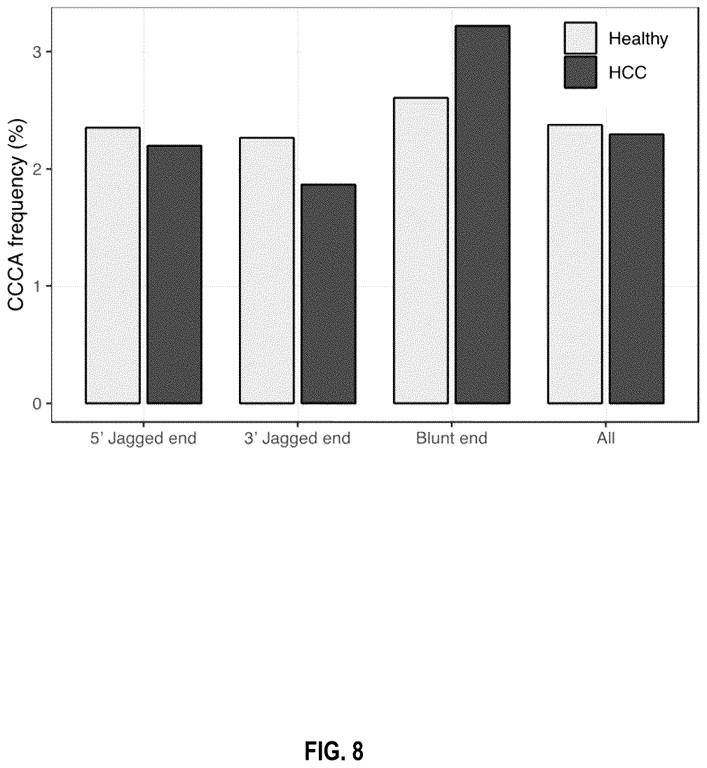

FIG. 8 is graph of the CCCA end motif across different types of ends. The y-axis shows the CCCA frequency as a percent. The x-axis shows where the CCCA end motif is found: 5′ protruding jagged end, 3′ protruding jagged end, blunt end, and all fragments. The two bars show the healthy case and the HCC case. Two-sided ends from one cfDNA fragment were analyzed separately.

As shown in FIG. 8, the frequency of 5′ CCCA end motif was decreased in the HCC case in the 5′ protruding jagged end or 3′ protruding jagged end of cfDNA. For the blunt end, the frequency of 5′ CCCA end motif was increased in the HCC case compared with the healthy subject. The difference in the frequency of 5′ CCCA end motif between the HCC case and the healthy subject in terms of the 5′ protruding jagged end, 3′ protruding jagged end, and blunt end was greater than 5′ CCCA end motif deduced from all fragment ends. FIG. 8 shows that determining the type of jagged end may increase accuracy of distinguishing HCC cases from healthy cases.

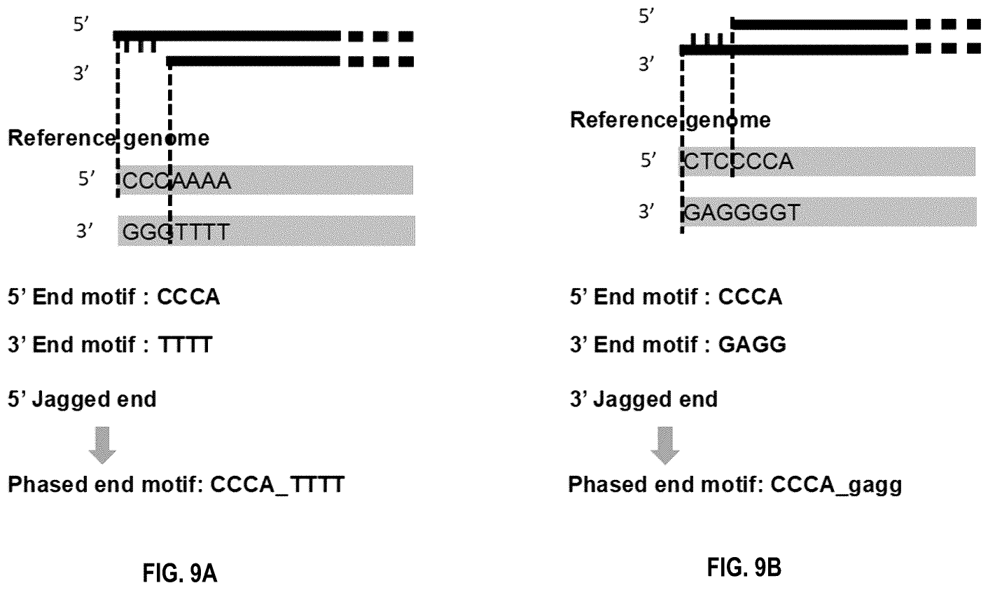

FIGS. 9A and 9B illustrate a technique that can combine the jagged end, 5′ end motif, and 3′ end motif to measure the phase of end modalities of cfDNA molecules. In FIG. 9A, there is a 5′ protruding jagged end with a 5′ “CCCA” end motif, and a 3′ “TTTT” end motif, the phase of end motif can be referred to as “CCCA_TTTT” where the 5′ end motif is followed by the 3′ end motif both of which are expressed in upper case letters with an underscore (i.e., “_”) as the separator.

In FIG. 9B, there is a 3′ protruding jagged end with a 5′ “CCCA” end motif, and a 3′ “GAGG” end motif, the phase of end motif can be referred as “CCCA_gagg”, where the 5′ end motif in upper case letters is followed by 3′ end motif in lower case letters. The lower case denotes that the 3′ end is the protruding end. Different naming conventions can be used to show the protruding end, the non-protruding end, and whether ends are blunt. In some embodiments, the end motif may include only nucleotides from one strand because the information about the other strand can be deduced from only one strand. A separator may be used to denote the location of the overhang. As an example, FIG. 9B may be represented by “3-GAG-GGGT”. The “3” denotes that the 3′ is protruding, and the 2^{nd} “−” denotes where the 5′ strand ends starts.

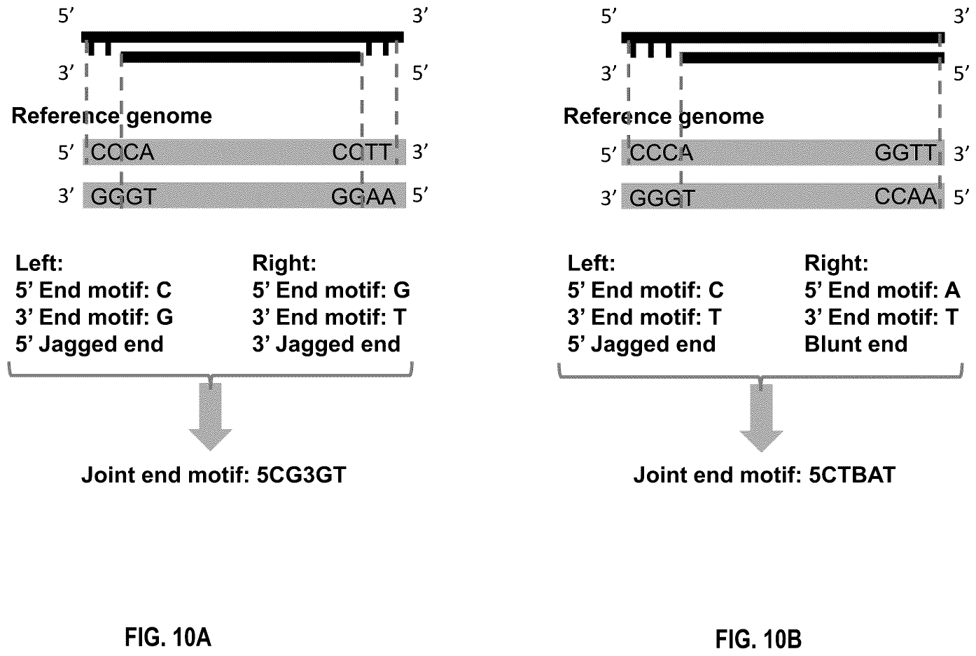

FIGS. 10A and 10B illustrate a technique that can combine the jagged end, 5′ end motif, and 3′ end motif from both sides of the fragments to measure the joint end modalities of cfDNA molecules. In FIG. 10A, there is a DNA fragment with a 5′ protruding jagged end with a 5′ “C” end motif, and a 3′ “G” end motif on the left and with a 3′ protruding jagged end with a 5′ “G” end motif, and a 3′ “T” end motif on the right. The joint end motif can be referred to as “5CG3GT” where the first 3 characters indicate the left end and following 3 characters indicate the right end.

In FIG. 10B, there is a DNA fragment with a 5′ protruding jagged end with a 5′ “C” end motif, and a 3′ “T” end motif on the left and with a blunt end with a 5′ “A” end motif, and a 3′ “T” end motif in the right. The joint end motif can be referred to as “5CTBAT” where the first 3 characters indicate the left end and following 3 characters indicate the right end. The 1^{st} letter of the 3 letters indicated the type of jagged ends, i.e., “5” for 5′ jagged end, “3” for 3′ jagged end, and “B” for blunt end. The 2^{nd} letter of the 3 letters indicated the 5′ end motif, and the 3^{rd} indicated the 3′ end motif.

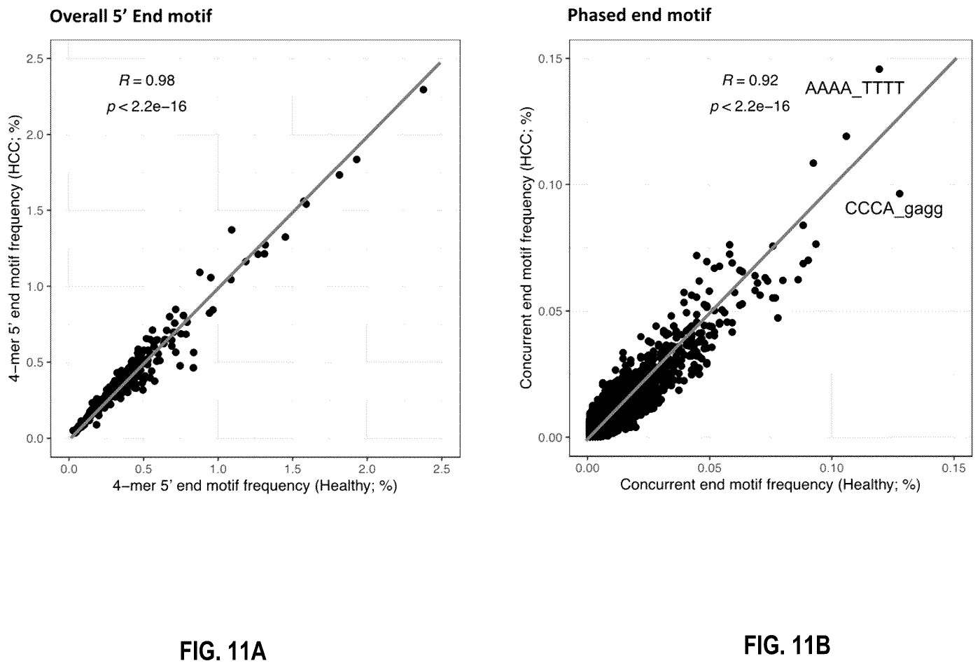

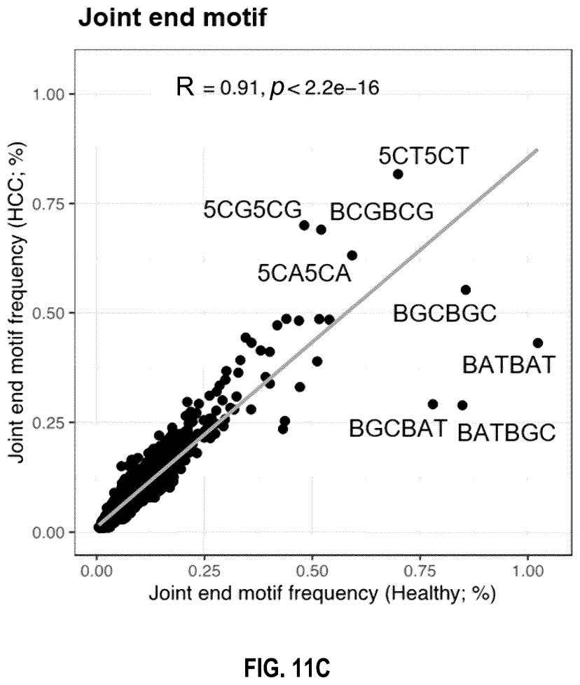

FIG. 11A is a graph of the correlation of the frequency of the overall 5′ end motif between HCC and healthy subjects. The y-axis shows the frequency of a 4-mer 5′end motif for HCC subjects. The x-axis shows the frequency of 4-mer 5′end motif for healthy subjects. Each dot represents a different 4-mer end motif. The data shows a high correlation with R=0.98 and p<2.2e-16. An end motif with dot that deviates farther from the line y-x may be useful in distinguishing HCC cases from healthy cases.

FIG. 11B is a graph of the correlation of the frequency of the phased end motifs between HCC and healthy subjects. The y-axis shows the frequency of a 4-mer concurrent end motif for HCC subjects. The x-axis shows the frequency of 4-mer concurrent end motif for healthy subjects. Each dot represents a different concurrent end motif including the 4-mers for both the 5′ end and the 3′ end, distinguishing between 5′ protruding ends and 3′ protruding ends. The data shows a correlation with R=0.92 and p<2.2e-16.

FIG. 11C is a graph of the correlation of the frequency of the joint end motifs between HCC and healthy subjects. The y-axis shows the frequency of joint end motifs for HCC subjects. The x-axis shows the frequency of joint end motifs for healthy subjects. Each dot represents a different joint end motif including the jagged end type, 1-mers motif for both the 5′ end and the 3′ end from both sides of the cfDNA fragments. The data shows a correlation with R=0.91 and p<2.2e-16.

Compared with typical analysis based on the overall 5′ end motifs in FIG. 11A, the phase of end motif showed a larger difference between HCC and healthy subjects in FIG. 111B. The rank of the top 4 motifs in overall 5′ end motifs was still the same between the HCC and healthy subjects. In contrast, the ranks of the top 4 phased end motifs were largely altered. For example, the top one phased end motif of the healthy subject (CCCA_gagg) went down to the 4^{th} in the HCC, whereas the ranked 2^{nd} phased end motif in the healthy subject (AAAA_TTTT) rose to the ranked 1′ phased end motif in the HCC patient. The differences in the phased end motif for HCC subjects and healthy subjects shows that different phased end motifs or combinations of different end motifs can be used to distinguish HCC cases from healthy cases.

Moreover, compared with the overall 5′ end motifs and the phased end motif, the joint motif further enlarged the difference between HCC and healthy subjects (FIG. 11C). The rank of the top 4 motifs in overall 5′ end motifs was the same between the HCC and healthy subjects. Although the ranks of the top 4 phased end motifs were largely altered, the top 4 phased end motifs were the same between the HCC (top 4 phased motif: AAAA_TTTT, CAAA_TTTT, CCCC_GGGT, and CCCA_gagg) and healthy subject (CCCA_gagg, AAAA_TTTT, CAAA_TTTT and CCCC_GGGT). In contrast, the top 4 joint end motifs were totally different between HCC (top 4 joint motif: 5CT5CT, 5CG5CG, BCGBCG, and 5CA5CA) and healthy subject (top 4 joint motif: BATBAT, BGCBGC, BATBGC and BGCBAT). The differences in the joint end motif for HCC subjects and healthy subjects shows that combinations of jagged end information, different end motifs from both side of cfDNA fragments can be used to distinguish HCC cases from healthy cases.

### B. Nuclease Activity

DNASEs play different roles in fragmentation of cfDNA. Jagged end modalities and/or end motifs may be used to analyze nuclease activity.

### 1. Jagged End Modalities

Our previous study indicated that different DNASEs play different roles in cfDNA jagged end generation. DNASE activity can be deduced by jagged ends (Ding et al. Clin Chem. 2022; 68:917-926). However, previously only the 5′ protruding jagged ends and not the 3′ protruding jagged ends were analyzed. Analysis of all types of jagged ends (e.g., 5′ protruding jagged, 3′ protruding jagged, and blunt end) and concurrent analysis of single-molecule end modalities may provide more information regarding the activity of different DNASEs.

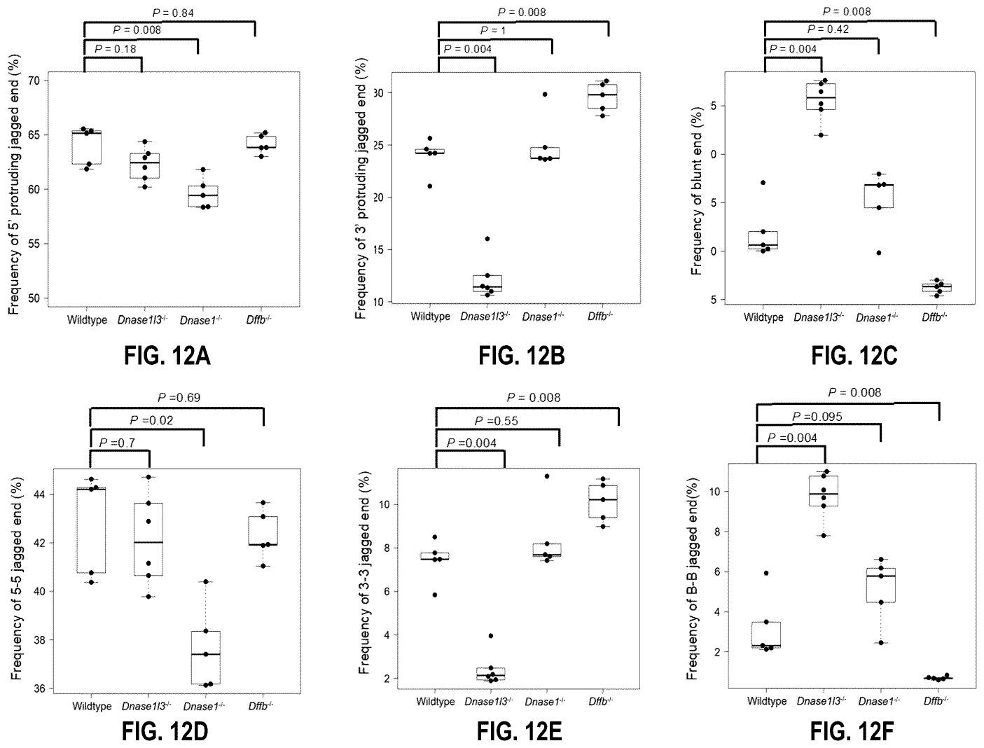

FIGS. 12A-12F show analysis of plasma cfDNA samples from wildtype, DNASE1 (DNASE1^{-/-}), DNASE1L3 (DNASE1L3^{-/-}), and DFFB (DFFB^{-/-}) knockout mouse models using analysis of single-molecule end modalities on the PacBio platform (median reads: 1,295,159; range: 176,285-2,624,708). The x-axis shows the category of nuclease activity. The y-axis shows the frequency of the particular jagged end modality.

DNASE^{-/-} mice indicated a significant decrease (8.76%) in the frequency of fragments carrying 5′ protruding jagged ends (FIG. 12A) and a significant reduction (52.80%) in the frequency of fragments carrying 3′ protruding jagged ends can be observed in DNASE1L3^{-/-} mice (FIG. 12B). A significant reduction (40.25%) in the frequency of fragments carrying blunt ends can be observed in DFFB^{-/-} mice (FIG. 12C). These results indicate that compared with the analysis of 5′ protruding jagged alone, analysis of all types of jagged ends can provide more information regarding the activity of different DNASEs.

We further categorized cfDNA fragments according to the jagged end modalities from both sides of a molecule (i.e., 5′ protruding jagged end+3′ protruding jagged end (5-3), 5′ protruding jagged end+5′ protruding jagged end (5-5), 3′ protruding jagged end+3′ protruding jagged end (3-3), 5′ protruding jagged end+blunt end (5-B), 3′ protruding jagged end+blunt end (3-B), blunt end+blunt end (B-B)). As shown in FIG. 12D, a greater decrease can be observed in the frequency of cfDNA fragments carrying 5-5 jagged ends compared with the frequency of 5′ protruding jagged ends in DNASE1^{-/-} mice (5-5 jagged ends vs 5′ protruding jagged end: 15.40% vs 8.76%). Similar to DNASE1−/− mice, a greater decrease can be observed in the frequency of cfDNA fragments carrying 3-3 jagged ends and B-B jagged ends compared with the frequency of 3′ protruding jagged ends and blunt ends in DNASE1L3−/− mice (decrease: 3-3 jagged ends vs 3′ protruding jagged end: 71.45% vs 52.80%) (FIG. 12E) and DFFB−/− mice (decrease: B-B jagged ends vs blunt end: 70.41% vs 40.25%) (FIG. 12F), respectively. These results indicated that concurrent analysis of jagged ends at both sides of one cfDNA fragment can improve the distinguishing power of different activities of different DNASEs. This technology can be used to enhance the diagnosis power of diseases with abnormal DNASE activities, such as but not limited to systemic lupus erythematosus.

### 2. End Motifs and Jagged End Modalities

Our previous publications reported that the cfDNA end motif could be used to deduce the activity of different DNASEs (Han et al. Am J Hum Genet. 2020; 106:202-214; Jiang et al. Cancer Discov. 2020; 10:664-673). The results discussed in the previous section indicated different DNASEs may be related to various types of jagged ends. Analyzing end motifs in different jagged end groups may improve the distinguishing power in detecting the change of DNASE activity.

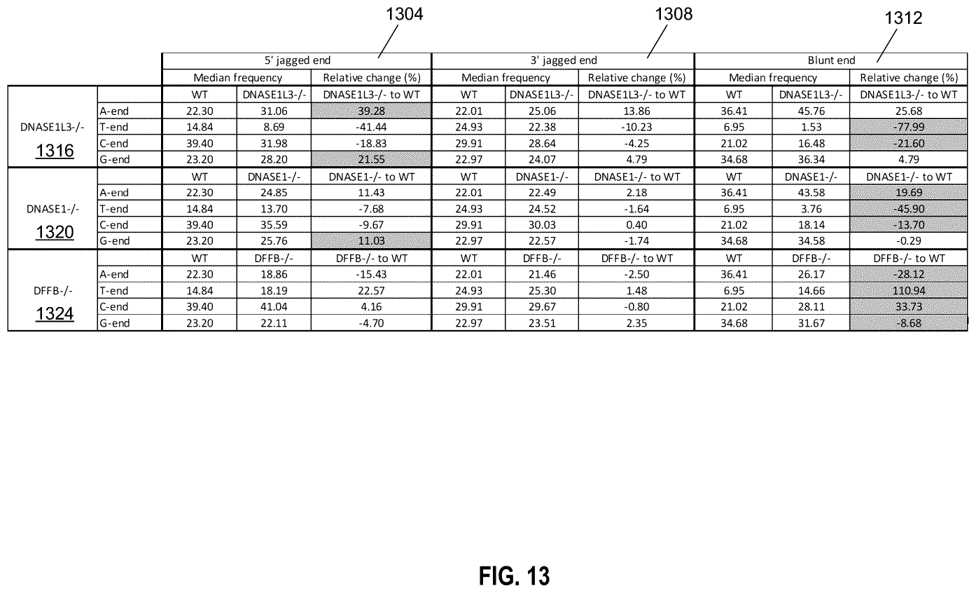

FIG. 13 is a table of the different jagged end modalities and end nucleotide type for different nuclease activities. Main columns 1304, 1308, and 1312 show data for different jagged ends. Main rows 1316, 1320, and 1324 show the different nuclease activities analyzed. The individual columns under each main column show median frequencies for wildtype mice and the particular nuclease knock out mice in the main row and a relative change in the median frequency between the nuclease knock out mice and wild type mice. The individual rows indicate the ending nucleotide. Cells shaded in gray indicate the greatest change of each ending nucleotide between different jagged end types. Each row has only one shaded cell. For example, for DNASE1L3−/− mice, greatest alteration of A-end was found in 5′ jagged end, so the well of relative changes of A-end in 5′ jagged end is shaded.

As shown in FIG. 13, compared with fragments with 3′ protruding jagged ends and fragments with blunt ends, fragments with 5′ protruding jagged ends showed the greatest increase of A-(median increase: 5′ vs 3′ vs blunt: 39.28% vs 13.86% vs 25.68%) and G-(median increase: 5′ vs 3′ vs blunt: 21.55% vs 4.79% vs 4.79%) 5′ end motif in DNASE1L3−/− mice compared with WT mice. Compared with fragments with 5′ protruding jagged ends and fragments with 3′ protruding jagged ends, fragments with blunt ends showed the greatest decrease of C-(median decrease: 5′ vs 3′ vs blunt: 18.83% vs 4.25% vs 21.60%), and T-(median decrease: 5′ vs 3′ vs blunt: 41.44% vs 10.23% vs 77.99%) 5′ end motif in DNASE1L3−/− mice compared with WT mice. In DNASE1−/− mice, compared with WT mice, fragments with blunt ends showed the greatest decrease of 5′ C-end (median decrease: 5′ vs 3′ vs blunt: 9.67% vs 0.40% vs 13.70%) and 5′ T-end (median decrease: 5′ vs 3′ vs blunt: 70.68% vs 1.64% vs 45.90%) and the most significant increase of 5′ A-end (median increase: 5′ vs 3′ vs blunt: 11.43% vs 2.18% vs 19.69%), while the 5′ protruding jagged ends showed the greatest increase of 5′ G-end (median increase: 5′ vs 3′ vs blunt: 11.03% vs −1.74% vs −0.29%). Interestingly, for DFFB^{-/-} mice, the greatest change of 5′ end motif have been observed in fragments with blunt ends (5′ C-end (median decrease: 5′ vs 3′ vs blunt: 4.16% vs −0.80% vs 33.73%); 5′ T-end (median decrease: 5′ vs 3′ vs blunt: 22.57% vs 1.48% vs 110.94%); 5′ A-end (median decrease: 5′ vs 3′ vs blunt: 15.43% vs 2.50% vs 28.12%); 5′ G-end (median decrease: 5′ vs 3′ vs blunt: 4.70% vs −2.35% vs 8.68%)). We further analyzed the 4-mer 5′ end motif in all fragments, fragments with 5′ protruding jagged ends, 3′ protruding jagged ends, and fragments with blunt ends in DFFB^{-/-} and WT mice.

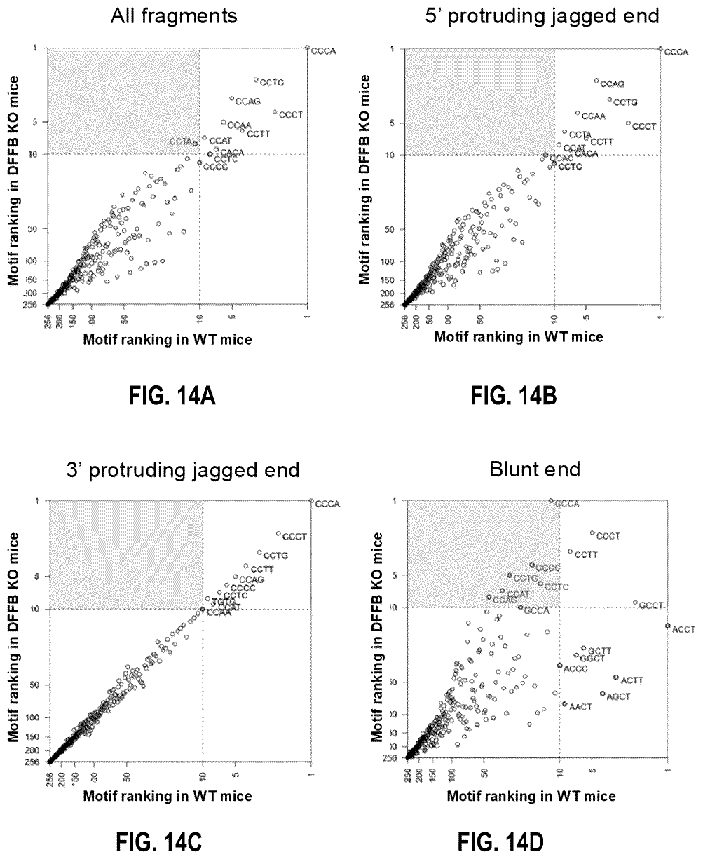

FIGS. 14A-14D are graphs of end motif rankings for DFFB^{-/-} (DFFB knockout [KO]) mice and wildtype (WT) mice. The figures have the motif ranking in wildtype mice on the x-axis, and the motif ranking in DFFB^{-/-} mice on the y-axis. FIG. 14A shows the 5′ end motif rankings of pooled all cfDNA fragments in DFFB^{-/-} mice and wildtype mice. FIG. 14B shows the 5′ end motif rankings of pooled cfDNA fragments carrying 5′ protruding jagged ends in DFFB^{-/-} mice and wildtype mice. FIG. 14C shows the 5′ end motif rankings of pooled cfDNA fragments carrying 3′ protruding jagged ends in DFFB^{-/-} mice and wildtype mice. FIG. 14D shows the 5′ end motif rankings of pooled cfDNA fragments carrying blunt ends in DFFB^{-/-} mice and wildtype mice. As shown in FIGS. 14A-14D, compared to all fragments, fragments with 5′ protruding jagged ends, and fragments with 3′ protruding jagged ends, the greatest difference between DFFB^{-/-} and WT mice can be observed in the fragments with blunt ends (R: all vs 5′ vs 3′ vs blunt: 0.94 vs 0.95 vs 1 vs 0.77). However, fragments with 5′ protruding jagged ends also have motifs that are either overrepresented or underrepresented in DFFB^{-/-} mice.

These data indicated that the concurrent analysis of single-molecule end modalities allows for more precisely deciphering of characteristic cleavages attributed to various DNA nucleases in plasma.

### C. Example Methods

Levels of a condition may be determined using any of the processes described herein. Example may include treating the disease or condition in the patient after determining the level of the disease or condition in the patient. Treatment may include any suitable therapy, drug, or surgery, including any treatment described in a reference mentioned herein. Information on treatments in the references are incorporated herein by reference.

Treatment can be provided according to a determined level of cancer, the identified mutations, and/or the tissue of origin. For example, an identified mutation (e.g., for polymorphic implementations) can be targeted with a particular drug or chemotherapy. The tissue of origin can be used to guide a surgery or any other form of treatment. And, the level of cancer can be used to determine how aggressive to be with any type of treatment, which may also be determined based on the level of cancer.

A statistically significant number of cell-free DNA molecules can be analyzed so as to provide an accurate determination the proportional contribution from the first tissue type. In some embodiments, at least 1,000 cell-free DNA molecules are analyzed. In some examples, at least 1,000 cell-free DNA molecules are analyzed. In other embodiments, at least 10,000 or 50,000 or 100,000 or 500,000 or 1,000,000 or 5,000,000 cell-free DNA molecules or more can be analyzed.

1. Jagged Ends from Concurrent Analysis

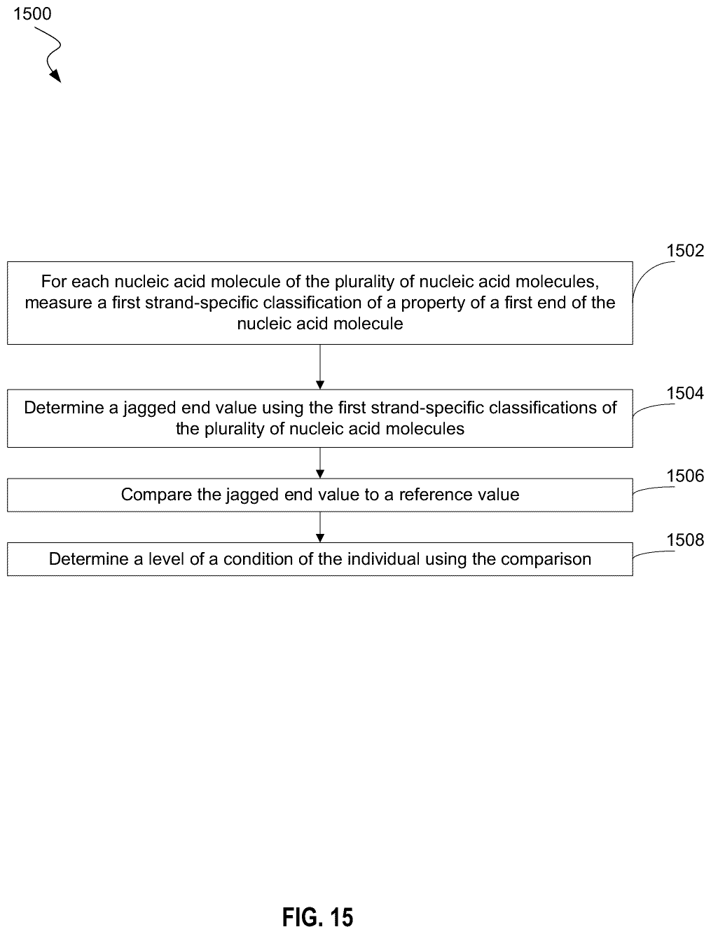

FIG. 15 is a flowchart of an example process 1500 of analyzing a biological sample obtained from an individual. The biological sample may include a plurality of nucleic acid molecules. The nucleic acid molecules may be cell-free and double-stranded with a first strand and a second strand. At least one of the nucleic acid molecules may have an overhang where the first strand or the second strand overlaps the other. Process 1500 may use overhang information from the four ends of the two strands of the nucleic acid molecules to determine a level of a condition of an individual. In some implementations, one or more process blocks of FIG. 15 may be performed by a system 10 or system 2400.

At block 1502, for each nucleic acid molecule of the plurality of nucleic acid molecules, a first strand-specific classification of a property of a first end of the nucleic acid molecule is measured. The strand-specific classification may indicate whether the first strand or the second strand overhangs the other, including if neither strand overhangs the other. The strand-specific classification may identify whether the first strand or the second strand is the 3′ strand or 5′ strand. The strand-specific classification may also indicate the length of an overhang of either the first strand or the second strand. The strand-specific classification may include the jagged end modality described herein. The property may be measured using process 300.

For each nucleic acid molecule of the plurality of nucleic acid molecules, a second strand-specific classification of the second end of the nucleic acid molecule may be measured.

At block 1504, a jagged end value is determined using the first strand-specific classifications of the plurality of nucleic acid molecules. The jagged end value may be an amount of nucleic acid molecules with a certain type of jagged end, including 5′ overhang, 3′ overhang, and blunt ends (e.g., FIG. 5A). The amount may be a number, a total length, a mass, or a frequency. In some embodiments, the jagged end value may be an amount of nucleic acid molecules including amounts from one of the following classifications: blunt-ended at the first end and blunt-ended at the second end, 5′ overhang at the first end and blunt-ended at the second end, 3′ overhang at the first end and blunt-ended at the second end, 5′ overhang at the first end and 3′ overhang at the second end, 5′ overhang at the first end and 5′ overhang at the second end, and 3′ overhang at the first end and 3′ overhang at the second end (e.g., FIG. 5B). Determining the jagged end value may use the second strand-specific classifications.

In some examples, the jagged end value may be an element in a vector. The vector may include a plurality of elements. The plurality of elements may include amounts of nucleic acid molecules in one or more of the following classifications: blunt-ended at the first end and blunt-ended at the second end, 5′ overhang at the first end and blunt-ended at the second end, 3′ overhang at the first end and blunt-ended at the second end, 5′ overhang at the first end and 3′ overhang at the second end, 5′ overhang at the first end and 5′ overhang at the second end, and 3′ overhang at the first end and 3′ overhang at the second end.

The plurality of elements may include a classification of nucleic acid molecules having sizes in one or more size ranges (e.g., FIGS. 5B and 5C). The one or more size ranges may be any size ranges described herein. The size ranges may include sizes less than or greater than any of the following sizes: 90, 100, 110, 120, 130, 140, 150, 160, 170, 180, 190, 200, 210, 220, 230, 240, 250, 260, 270, 280, 290, 300, 310, 320, 330, 340, or 350. Additionally, the size ranges may include sizes between and including any two sizes described herein. The size of each nucleic acid molecule may be determined by aligning subsequences corresponding to the ends of the respective nucleic acid molecule with a reference genome.

The jagged end value may include a ratio of amounts of nucleic acid molecules in one overhang classification and having sizes in a certain size range and amounts of nucleic acid molecules with the same overhang classification having sizes in a different size range. The vector may include size ratios for each of a plurality of overhang classifications (e.g., FIG. 7A).

At block 1506, the jagged end value is compared to a reference value. The comparison may determine whether the jagged end value is statistically significantly different from the reference value. The reference value may be any reference value described herein. The vector may be compared to a reference vector (which may include a plurality of different reference values). The comparison may be between corresponding elements in the vectors. In some embodiments, the comparison may be performed by a machine learning model. For example, a machine learning model may be trained using jagged end values determined from subjects with known levels of condition.

At block 1508, a level of a condition of the individual is determined using the comparison. The condition may be cancer, an autoimmune disease, a pregnancy-associated disorder, nuclease activity deficiency, or any condition described herein. The reference value may be determined from one or more subjects having a certain level of the condition or one or more healthy subjects. If the jagged end value is statistically the same as the reference value, then the level of the condition may be determined to be the same as the subject or subjects associated with the reference value.

In some examples, the level of the condition is not determined. Instead, a fractional concentration of clinically-relevant DNA may be determined using the comparison. The reference value may be determined from one or more subjects having a known fractional concentration of clinically-relevant DNA. The reference value may be a calibration value determined using a calibration sample.

Process 1500 may include additional implementations, such as any single implementation or any combination of implementations described herein and/or in connection with one or more other processes described elsewhere herein.

Although FIG. 15 shows example blocks of process 1500, in some implementations, process 1500 may include additional blocks, fewer blocks, different blocks, or differently arranged blocks than those depicted in FIG. 15. Additionally, or alternatively, two or more of the blocks of process 1500 may be performed in parallel.

### 2. End Motifs at One End

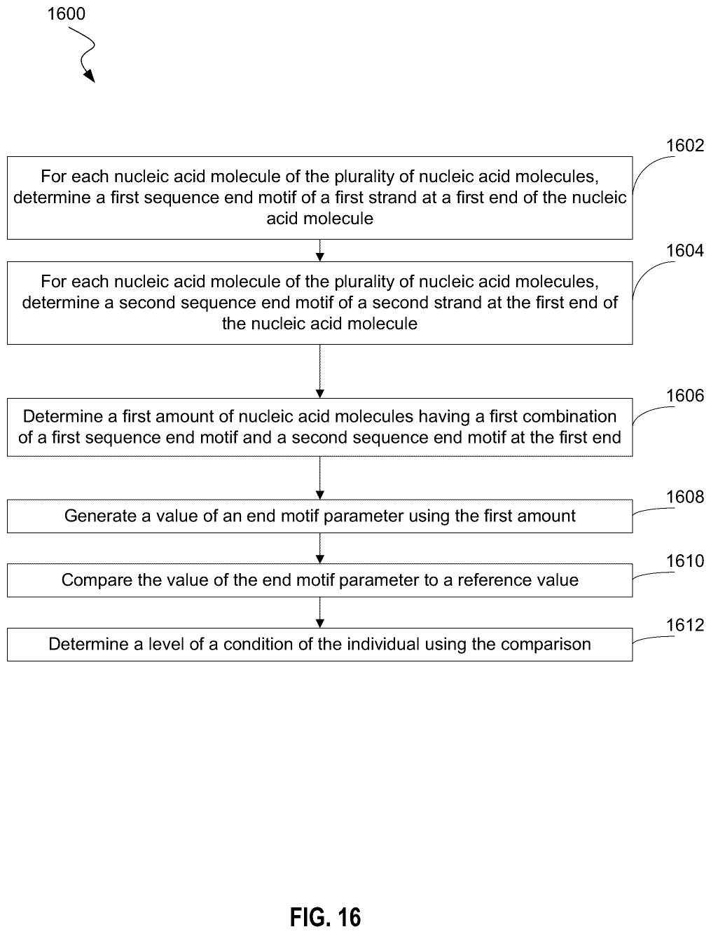

FIG. 16 is a flowchart of an example process 1600 of analyzing a biological sample obtained from an individual. The biological sample may include a plurality of nucleic acid molecules. The nucleic acid molecules may be cell-free and double-stranded with a first strand and a second strand. Process 1600 may use the end motifs at least at one end of a molecule to determine the level of a condition. At least some of the nucleic acid molecules of the plurality of nucleic acid molecules having nucleotides on one strand that have no complementary portion on the other strand. In some implementations, one or more process blocks of FIG. 16 may be performed by a system 10 or system 2400.

At block 1602, for each nucleic acid molecule of the plurality of nucleic acid molecules, a first sequence end motif of the first strand at a first end of the nucleic acid molecule is determined.

At block 1604, a second sequence end motif of the second strand at the first end of the nucleic acid molecule is determined. In examples, the first strand may have the 5′ end at the first end. In other examples, the first strand may have the 3′ end at the first end. The first strand may overhang the second strand, or the second strand may overhang the first strand. The first end may be a blunt end. The subsequences may be determined using process 300. In some embodiments, the second sequence end motif of the second strand may be determined by taking the complementary nucleotides of the corresponding nucleotides in the first strand.

At block 1606, a first amount of nucleic acid molecules having a first combination of the first sequence end motif and the second sequence end motif at the first end is determined. The sequence end motifs may have 2, 3, 4, 5, 6, 7, 8, 9, or 10 nucleotides. The amount may be a number, a total length, a mass, or a frequency. The first combination may be the phased end motifs described with FIG. 9A and FIG. 9B.

At block 1608, a value of an end motif parameter is generated using the first amount. In some embodiments, the end motif parameter may be the first amount. In some embodiments, the end motif parameter may a ratio of the first amount to other amounts (e.g., amounts of all end motifs). As an example, the end motif parameter may be a frequency.

A second phased end motif may be used in addition to the first phased end motif. A second amount of nucleic acid molecules having a second combination of a third sequence end motif and a fourth sequence end motif at the first end may be determined. The third sequence end motif may be at the 5′ strand or the 3′ strand. As an example, the first amount may be the amount of AAAA_TTTT and the second amount may be the amount of CCCA_gagg. Generating the value of the end motif parameter may use the second amount. The end motif parameter may be a vector of different amounts of certain combinations of end motifs.

At block 1610, the value of the end motif parameter is compared to a reference value. The reference value may be any reference value described herein. The comparison may be any comparison described herein, including with block 1506.

At block 1612, a level of a condition of the individual is determined using the comparison. The determination may be performed similar to block 1508.

The condition may be cancer, HCC, an autoimmune disease, a pregnancy-associated disorder, a nuclease activity deficiency, or any condition described herein. The reference value may be determined from one or more subjects having a certain level of the condition or one or more healthy subjects.

In some examples, the level of the condition is not determined. Instead, a fractional concentration of clinically-relevant DNA may be determined using the comparison. The reference value may be determined from one or more subjects having a known fractional concentration of clinically-relevant DNA. The reference value may be a calibration value determined using a calibration sample.

Process 1600 may include using the two sequence motifs from the jagged ends at the other end of molecules. Four sequence motifs from the same molecule may be used. In embodiments, the plurality of nucleic acid molecules is a first plurality of nucleic acid molecules. The biological sample may include a second plurality of nucleic acid molecules. The first plurality of nucleic acid molecules may include a subset of the second plurality of nucleic acid molecules. Process 1600 may further include for each nucleic acid molecule of a second plurality of nucleic acid molecules, determining a third sequence end motif on the first strand at a second end of the nucleic acid molecule without a complementary portion on the second strand, and determining a fourth sequence end motif on the second strand at the second end of the nucleic acid molecule. A second amount of nucleic acid molecules having a second combination of a third sequence end motif and a fourth sequence end motif at the second end may be determined. The value of the end motif parameter may be generated using the second amount. In some embodiments, the value of the end motif parameter may be the amount of molecules having a certain combination of four sequence motifs present on the molecule.

In some embodiments, the jagged end modality at one end may also be used for determining the level of a condition. Process 1600 may include for each nucleic acid molecule of the plurality of nucleic acid molecules, measuring a first strand-specific classification of a property of a first end of the nucleic acid molecule. The strand-specific classification may indicate whether the first strand or the second strand overhangs the other. For example, at one end, the strand-specific classification may indicate a 3′ protruding end, a 5′ protruding end, a blunt end, or a jagged end (generally). Determining the first amount may include determining the first amount of nucleic acid molecules having the first combination and the first strand-specific classification.

In some embodiments, the jagged end modality of the second end may also be used. Process 1600 may include for each nucleic acid molecule of the plurality of nucleic acid molecules, measuring a second strand-specific classification of the second end of the nucleic acid molecule. Determining the first amount may include determining the first amount of nucleic acid molecules having the first combination, the first strand-specific classification, and the second strand-specific classification.

Process 1600 may include additional implementations, such as any single implementation or any combination of implementations described herein and/or in connection with one or more other processes described elsewhere herein.

Although FIG. 16 shows example blocks of process 1600, in some implementations, process 1600 may include additional blocks, fewer blocks, different blocks, or differently arranged blocks than those depicted in FIG. 16. Additionally, or alternatively, two or more of the blocks of process 1600 may be performed in parallel.

### 3. End Motif at 3′ End

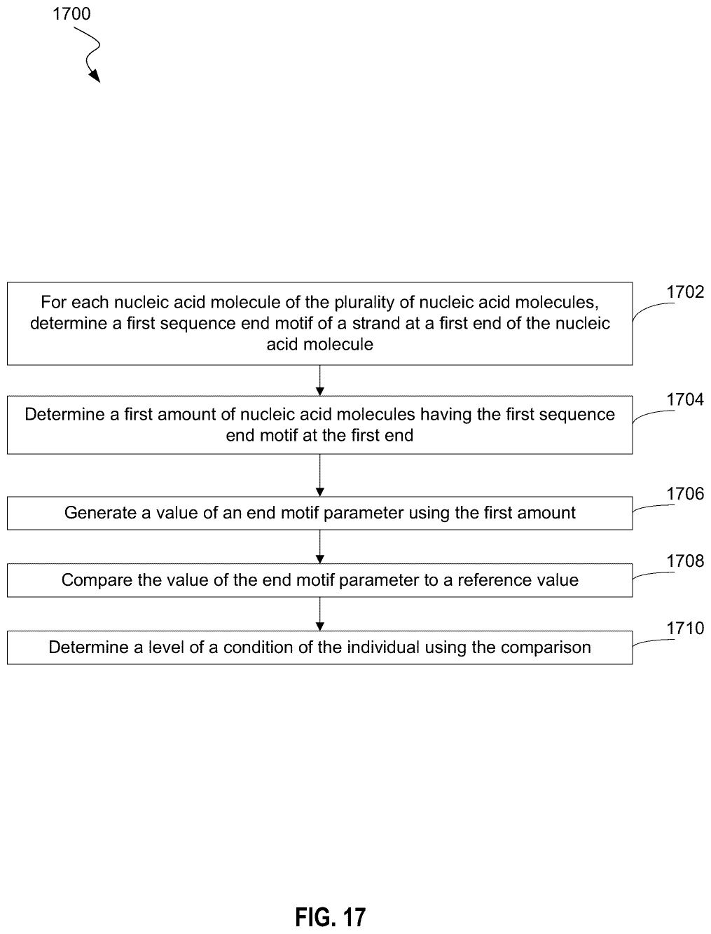

FIG. 17 is a flowchart of an example process 1700 of analyzing a biological sample obtained from an individual. The biological sample may include a plurality of nucleic acid molecules. The nucleic acid molecules may be cell-free and double-stranded with a first strand and a second strand. Process 1700 may use the end motifs at least at one end of a molecule to determine the level of a condition. At least some of the nucleic acid molecules of the plurality of nucleic acid molecules having nucleotides on one strand that have no complementary portion on the other strand. In some implementations, one or more process blocks of FIG. 17 may be performed by a system 10.

At block 1702, for each nucleic acid molecule of the plurality of nucleic acid molecules, a first sequence end motif of a strand at a first end of the nucleic acid molecule is determined, where the first end is the 3′ end for the strand. The first sequence end motif is the actual end motif of the original molecule rather than an end motif of a 3′ end after the molecule is blunt ended, either by filling in nucleotides on the 3′ strand or by removing nucleotides on the 3′ strand. The first sequence end motif may be determined using process 300.

At block 1704, a first amount of nucleic acid molecules having the first sequence end motif at the first end is determined. The first amount may be an absolute or relative amount.

The sequence end motifs of both ends of a single strand may be determined. In some examples, process 1700 may include for each nucleic acid molecule of the plurality of nucleic acid molecules, determining a second sequence end motif of the strand at a second end of the nucleic acid molecule. The first amount is of nucleic acid molecules having the first sequence motif at the first end and the second sequence end motif at the second end.

In some embodiments, the size of a single strand may be determined. Both ends of the single strand may be aligned to a reference genome to determine the size. The size of the complementary strand may also be determined. The length difference between the two strands may be generated using the two sizes. A statistical value of a length difference between the two strands for a plurality of molecules may be determined and compared to a reference value. The comparison may be used to determine a level of a condition. The length difference may also be determined by adding or subtracting the lengths of the overhangs at each end of a molecule, without ever needing to determine the length of either strand.

At block 1706, a value of an end motif parameter is generated using the first amount. In some embodiments, the end motif parameter may a ratio of the first amount to other amounts (e.g., amounts of all end motifs). As an example, the end motif parameter may be a frequency.

At block 1708, the value of the end motif parameter is compared to a reference value. The reference value may be any reference value described herein. For example, the reference value may be determined from a calibration sample, having a known level of a condition of the individual.

At block 1710, a level of a condition of the individual is determined using the comparison.

In some embodiments, the end motifs of both 3′ ends of a single molecule may be used. The strand may be a first strand. The end motif parameter may be a first end motif parameter. The reference value may be a first reference value. Process 1700 may further include for each nucleic acid molecule of the plurality of nucleic acid molecules, determining a second sequence end motif of a second strand at a second end of the nucleic acid molecule. The second end is the 3′ end for the second strand. Process 1700 may include determining a second amount of nucleic acid molecules having the second sequence end motif at the second end. A value of a second end motif parameter may be generated using the second amount. The value of the second end motif parameter may be compared to a second reference value. The level of the condition may be determined using the comparison.

In some embodiments, the jagged end modality at one end may also be used for determining the level of a condition. Process 1700 may include for each nucleic acid molecule of the plurality of nucleic acid molecules, measuring a first strand-specific classification of a property of a first end of the nucleic acid molecule. A strand-specific classification may indicate whether the first strand or the second strand overhangs the other. For example, at one end, the strand-specific classification may indicate a 3′ protruding end, a 5′ protruding end, a blunt end, or a jagged end (generally). The first (or second) strand-specific classification may be a specific jagged end modality from the list of possible strand-specific classification. Determining the first amount may include determining the first amount of nucleic acid molecules having the first combination and the first strand-specific classification.

In some embodiments, the jagged end modality of the second end may also be used. Process 1700 may include for each nucleic acid molecule of the plurality of nucleic acid molecules, measuring a second strand-specific classification of the second end of the nucleic acid molecule. Determining the first amount may include determining the first amount of nucleic acid molecules having the first combination, the first strand-specific classification, and the second strand-specific classification.

Process 1700 may include additional implementations, such as any single implementation or any combination of implementations described herein and/or in connection with one or more other processes (e.g., process 1600) described elsewhere herein.

Although FIG. 17 shows example blocks of process 1700, in some implementations, process 1700 may include additional blocks, fewer blocks, different blocks, or differently arranged blocks than those depicted in FIG. 17. Additionally, or alternatively, two or more of the blocks of process 1700 may be performed in parallel.

### III. Enrichment

Certain types of DNA, including clinically-relevant DNA, may tend to be more greatly represented among DNA with certain jagged end modalities or sequence end motifs. Accordingly, enriching for certain jagged end modalities and/or sequence end motifs may result in a sample that is enriched for certain types of clinically-relevant DNA. Enriching may include physical enriching of a sample or in silico enrichment of reads obtained from analyzing a biological sample.

### A. Jagged Ends

To explore the potential applications of concurrent analysis of single-molecule end modalities in non-invasive prenatal testing (NIPT), the end modalities have been analyzed in fetal-specific and shared cfDNA fragments in maternal plasma. Fetal-specific and shared cfDNA fragments were defined by the genotypes regarding the maternal buffy coat and placenta tissue samples, which were obtained using microarray-based genotyping technology (HumanOmni2.5 genotyping array Illumina). Informative SNPs were identified (i.e., where the mother was homozygous (denoted as AA genotype), and the fetus was heterozygous (denoted as AB genotype)). Fetal-specific DNA fragments were identified according to the DNA fragments carrying fetal-specific alleles at informative SNP sites. In this scenario, the B allele was fetal-specific, and the DNA fragments carrying the B allele were deduced to be originated from fetal tissues. Shared DNA fragments were identified according to the DNA fragments carrying shared alleles at informative SNP sites. In this scenario, the A allele was shared, and the DNA fragments carrying the A allele were deduced to originate from fetal and maternal tissues (mainly from maternal tissues). The number of fetal-specific molecules (p) carrying the fetal-specific alleles (B) was determined. The number of molecules (q) carrying the shared alleles (A) was determined. The fetal DNA fraction across all cell-free DNA samples would be calculated by 2p/(p+q)*100%.

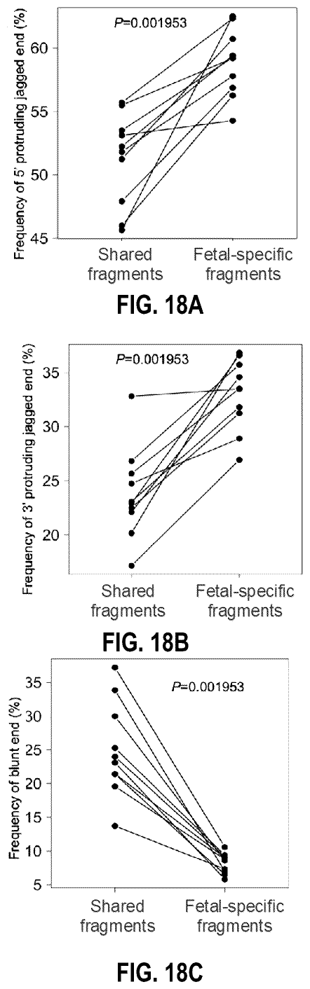

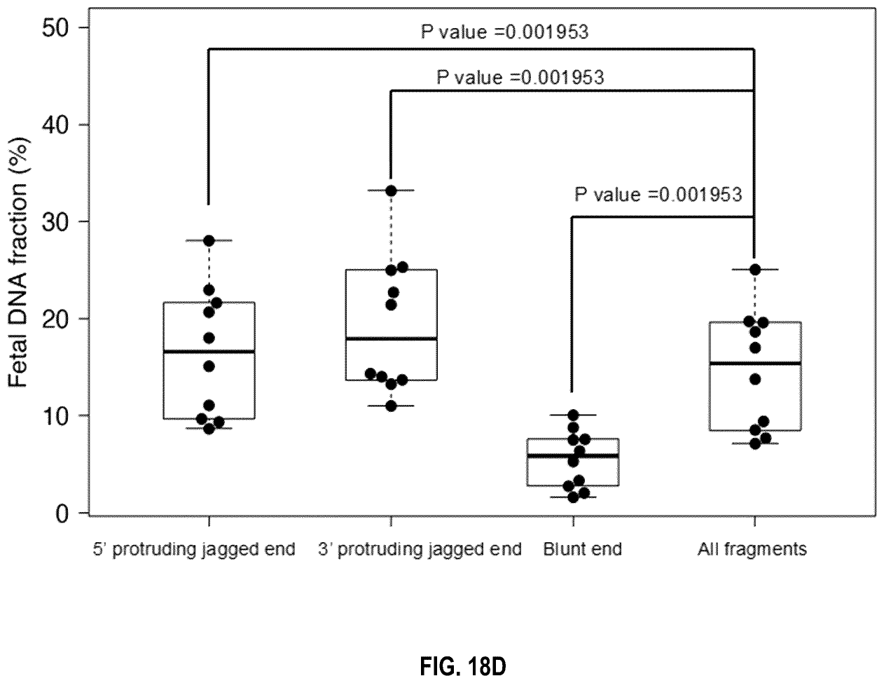

FIGS. 18A-18C shows the concurrent analysis of single-molecule end modalities on PacBio sequencing platform to a total of 10 plasma DNA samples of pregnant women (median number of reads: 1,305,115; Range: 393,197-1,921,070).

FIG. 18A is a graph of the frequency of 5′ protruding jagged ends. The y-axis shows the frequency of 5′ protruding jagged ends in percent. The x-axis shows fragments carrying shared alleles and fragments carrying fetal-specific alleles. Compared with shared cfDNA, fetal-specific cfDNA carry more 5′ protruding jagged ends (shared vs. fetal-specific: median: 52.0% vs. 59.2%).

FIG. 18B is a graph of the frequency of 3′ protruding jagged ends. The y-axis shows the frequency of 3′ protruding jagged ends in percent. The x-axis shows fragments carrying shared alleles and fragments carrying fetal-specific alleles. Compared with shared cfDNA, fetal-specific cfDNA carry more 3′ protruding jagged ends (shared vs. fetal-specific: median: 23.0% vs. 33.5%).

FIG. 18C is a graph of the frequency of blunt ends. The y-axis shows the frequency of blunt ends in percent. The x-axis shows fragments carrying shared alleles and fragments carrying fetal-specific alleles. Compared with shared cfDNA, fetal-specific cfDNA carry fewer blunt ends (shared vs. fetal-specific: median: 23.5% vs. 8.7%).

FIG. 18D shows fetal DNA fraction percentages based on different end modalities. The y-axis shows the fetal DNA fraction as a percent. The x-axis shows the different end modalities. Selective analysis of cfDNA carrying 5′ protruding jagged ends (5′ protruding jagged ends vs. all fragments: median: 16.61% vs. 15.41%) or 3′ protruding jagged ends (3′ protruding jagged ends vs. all fragments: median: 17.94% vs. 15.41%) showed a significant increase in fetal DNA fraction compared to all cfDNA fragments. In contrast, selective analysis of cfDNA carrying blunt ends (blunt ends vs. all fragments: median: 5.88% vs. 15.41%) showed a significant decrease in fetal DNA fraction compared to all cfDNA fragments, which indeed indicated a significant increase in DNA fraction of maternal origin. FIGS. 18A-18D show that the type of jagged end can be used to enrich DNA of a specific origin.

In another embodiment, cfDNA fragments were categorized into 6 different groups according to the jagged end modalities from both sides of the molecule (e.g., 5′ protruding jagged end+3′ protruding jagged end (5-3), 5′ protruding jagged end+5′ protruding jagged end (5-5), 3′ protruding jagged end+3′ protruding jagged end (3-3), 5′ protruding jagged end+blunt end (5-B), 3′ protruding jagged end+blunt end (3-B), blunt end+blunt end (B-B)).

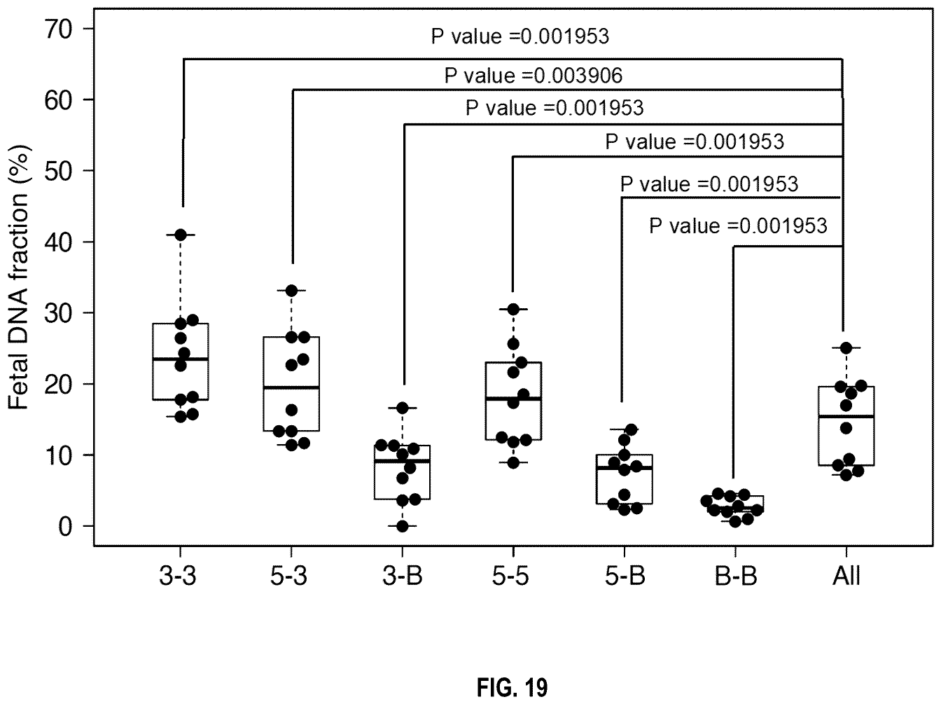

FIG. 19 is a graph of fetal DNA fraction versus different jagged end modalities. The y-axis shows the fetal DNA fraction deduced from cfDNA fragments. The x-axis shows the different end modalities (e.g., the 5′ protruding jagged end and 3′ protruding jagged end (5-3), 5′ protruding jagged end and 5′ protruding jagged end (5-5), 3′ protruding jagged end and 3′ protruding jagged end (3-3), 5′ protruding jagged end and blunt end (5-B), 3′ protruding jagged end and blunt end (3-B), blunt end and blunt end (B-B)) and all fragments. Selective analysis of cfDNA belonging to the 3-3 (3-3 vs. all fragments: median: 23.48% vs. 15.41%), 5-3 (5-3 vs. all fragments: median: 19.50% vs. 15.41%), and 5-5 (5-5 vs. all fragments: median: 17.93% vs. 15.41%) groups showed a significant increase in fetal DNA fraction compared to all cfDNA fragments. In contrast, selective analysis of cfDNA belonging to the 3-B (3-B vs. all fragments: median: 9.12% vs. 15.41%), 5-B (5-B vs. all fragments: median: 8.17% vs. 15.41%), and B-B (B-B vs. all fragments: median: 2.53% vs. 15.41%) groups showed a significant decrease in fetal DNA fraction compared to all cfDNA fragments. These results indicate that the analysis of jagged end modalities from both sides of a cfDNA fragment can be enriched for clinically-relevant DNA.

### B. Concurrent Analysis of Jagged Ends and End Motifs

Concurrent analysis of single-molecule end modalities (e.g., combining end motif with jagged end) can enrich fetal DNA in maternal plasma. We pooled together all sequenced reads of 10 pregnant women mentioned above (reads: 12,142,332).

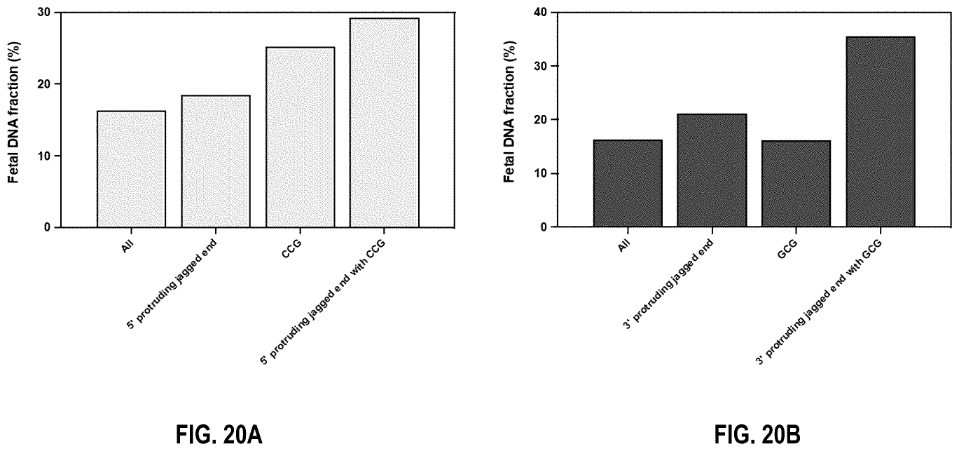

FIGS. 20A and 20B are graphs of the fetal DNA fraction deduced from fragments with certain jagged end modalities and sequence end motifs. The y-axis of the graphs is the fetal DNA fraction deduced from cfDNA fragments. The x-axis shows different categories of fragments: all fragments, the jagged end modality, the end motif, and the combination of the jagged end modality and the end motif.

FIG. 20A shows fragments carrying a 5′ protruding jagged end together with a 5′ CCG end motif on either side of the fragments (fetal DNA fraction: 29.3%) with a substantial increase in the fetal DNA fraction compared with all fragments (fetal DNA fraction: 16.3%), fragments with 5′ protruding jagged end only (fetal DNA fraction: 18.5%), or fragments with CCG 5′ end motif only (fetal DNA fraction: 25.2%).

FIG. 20B shows fragments carrying a 3′ protruding jagged end together with a 5′ GCG end motif on either side of the fragments (fetal DNA fraction: 35.6%) with a substantial increase in the fetal DNA fraction compared with all fragments (fetal DNA fraction: 16.3%), fragments with 3′ protruding jagged end only (fetal DNA fraction: 21.2%), or fragments with GCG 5′ end motif only (fetal DNA fraction: 16.2%). These results indicate that concurrent analysis of end motif with jagged end in the same end can facilitate the enrichment of fetal DNA in maternal plasma. Concurrent analysis of single-molecule end modalities can facilitate NIPT.

### C. Example Methods

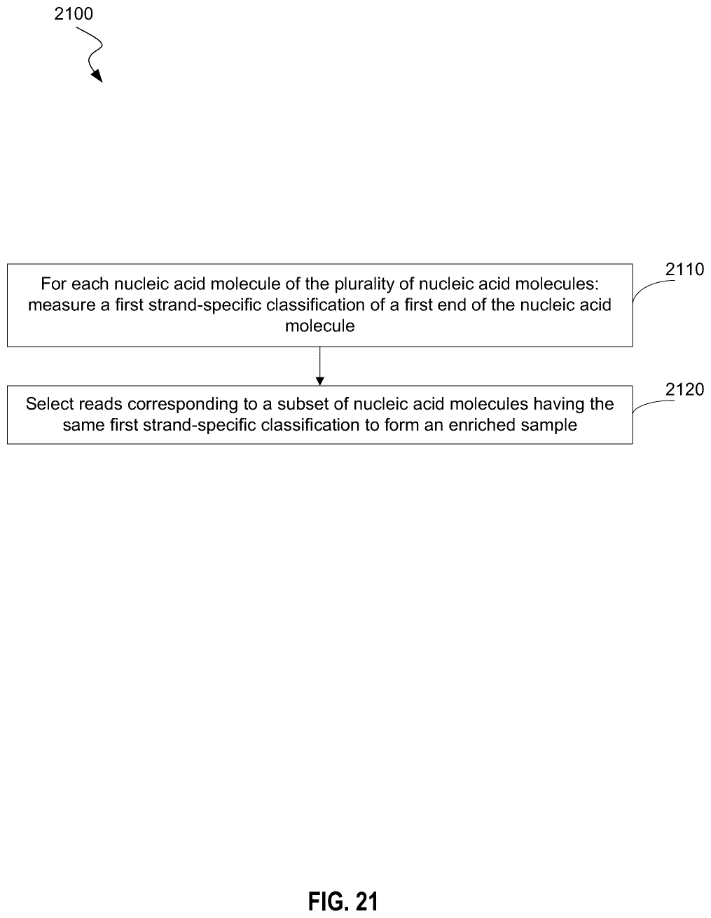

FIG. 21 is a flowchart of an example process 2100 for enriching a biological sample for clinically-relevant DNA. The biological sample may include the clinically-relevant DNA and other DNA. Each nucleic acid molecule of the plurality of nucleic acid molecules is double-stranded with a first strand and a second strand. The clinically-relevant DNA may be tumor DNA, transplant DNA, or fetal DNA. The biological sample may be obtained from a female subject pregnant with a fetus, and the clinically-relevant DNA may be either fetal DNA or maternal DNA. In some implementations, one or more process blocks of FIG. 21 may be performed by a system 2400.

At block 2110, a first strand-specific classification of a first end of the nucleic acid molecule is measured for each nucleic acid of the plurality of nucleic acid molecules. The strand-specific classification indicates whether the first strand or the second strand overhangs the other strand. The strand-specific classification may include the first strand overhanging the second strand, the second strand overhanging the first strand, and/or neither strand overhanging the other (blunt end). The subset of nucleic acid molecules may have the first strand-specific classification be the first strand overhanging the second strand. The first strand may be the 3′ strand or the 5′ strand. In some embodiments, the first strand-specific classification of the subset of nucleic acid molecules indicates the first strand of the nucleic acid molecule overhangs the second strand, and the second strand-specific classification of the subset of nucleic acid molecules indicates the second strand of the nucleic acid molecule overhangs the first strand. For example, the 5′ end may overhang at both ends.

At block 2120, reads corresponding to a subset of nucleic acid molecules having the first strand-specific classification are selected to form an enriched sample. The enriched sample may be an enriched in silico sample. In some embodiments, the enriched sample may be formed through physical enrichment techniques. For example, jagged end specific hybridization based targeted capture for enriching a certain number of jagged ends of interest may be used, in accordance with some embodiments. In one embodiment for physical enrichment analysis, one could use jagged end specific hybridization based targeted capture for enriching the jagged ends of interest. Biotinylated RNA probes which could be specifically hybridized to the jagged ends of interest were designed. The jagged ends of interest which would be hybridized with biotinylated probes could be pulled down by the streptavidin-coated magnetic beads. The RNA probes would be degraded by ribonucleases such as RNase H. The jagged ends of interest would be enriched in the pull-down material. In one embodiment, one or more different jagged ends were analyzed together, e.g., ratios or deviations between readouts of different jagged ends for practical applications.

In some embodiments, a second strand-specific classification of the second end of the nucleic acid molecule for each nucleic acid molecule of the plurality of nucleic acid molecules. The subset of nucleic acid molecules may have the second strand-specific classification. For example, the enriched sample may include molecules having the same type of overhang at one end and the same type of overhang at the other end.

In embodiments, a first sequence end motif at the first end of the nucleic acid molecule may be determined for each nucleic acid molecule of the plurality of nucleic acid molecules. Selecting the reads corresponding to the subset of the nucleic acid molecules may include selecting reads corresponding to nucleic acid molecules having the first sequence end motif.

In embodiments, a second sequence end motif at the second end of the nucleic acid molecule may be determined for each nucleic acid molecule of the plurality of nucleic acid molecules. Selecting the reads corresponding to the subset of nucleic acid molecules may include selecting reads corresponding to nucleic acid molecules having the same second sequence end motif.

In some embodiments, the method may further include analyzing the subset of nucleic acid molecules to determine a classification of a level of a disorder. For example, the methods may include aligning the reads of the subset to a reference genome. Methylation-aware sequencing or other detection technique may be performed to determine a methylation level or methylation pattern (e.g., methylation statuses at one or more genomic sites). The methylation level or methylation pattern may be compared to a reference level or pattern of a control sample having a known level of the disorder. The level of the disorder may be determined using the comparison.

In some embodiments, the method may include determining a chromosomal aberration or a fetal haplotype. The reads of the subset may be aligned to a reference genome. Chromosomal aberrations (e.g., amplifications or deletions) or a fetal haplotype may be identified from the alignment.

In embodiments, process 2100 may further include determining a first amount of the reads. A first parameter may be determined using the first amount of the reads. The first parameter may be determined using the first amount and another amount of sequence reads (e.g., total amount of reads or reads with a certain strand-specific classification or sequence end motif). In some examples, both of such amounts can be separate parameters. The other amount can take various forms, e.g., corresponding to a total number of sequence reads and/or DNA molecules analyzed. The first parameter may be a ratio of the amounts.

A characteristic of the biological sample may be determined using the first parameter. The first characteristic may be a fractional concentration of clinically-relevant DNA molecules in the biological sample. The characteristic of the biological sample may be a level of abnormality in the biological sample. A first value for the characteristic of the biological sample is estimated by comparing the first parameter to one or more calibration values determined from one or more calibration samples whose values for the characteristic are known.

Parameters generated based on respective nucleases can thus be used to determine the characteristic of the biological sample These respective parameters can be combined to form a new combined parameter, e.g., as a ratio, a ratio of respective functions of the respective parameters, and as two inputs to more complex functions, such as a machine learning model. Example combined parameters can include DNASE1L3/DFFB, DNASE1/DFFB, or other ratios of DNASE1L3:DNASE1:DFFB. Further, the parameters of more than two nucleases can be used, e.g., relative parameters of 3 or more nucleases can be used.

In some embodiments, the first value for the characteristic of the biological sample is estimated based on analyzing a set of parameters, in which each parameter corresponds to an amount of sequence reads that each include an ending sequence corresponding to a particular sequence end signature in combination with another amount (e.g., for normalization). For instance, a parameter can include a particular combination of frequency ratios between two sets of sequence reads with their respective end signatures. For example, a first parameter of the set of parameters may correspond to a ratio of strand-specific classifications between a first amount of sequence reads each including a strand-specific classification corresponding to a strand-specific classifications of a first nuclease and another amount of sequence reads, and a second parameter of the set of parameters may correspond to a ratio of strand-specific classifications between a second amount of sequence reads each including a strand-specific classifications corresponding to an end signature of a second nuclease and a third amount of sequence reads. In some instances, the third amount of sequence reads is the other amount of sequence reads used to determine the first parameter.

The determined characteristic can include a gestational age or range (e.g., 8 weeks, or 9-12 weeks), e.g., when a nuclease is differentially regulated between fetal tissue and maternal tissue. In another example, the determined characteristic can be a particular tissue type (e.g., liver cells) relative to the other tissue type (e.g., hematopoietic cells). The characteristic of the target tissue type may also indicate a particular condition of the target tissue type (e.g., HCC, preeclampsia, preterm birth). In another example, the determined characteristic can be a size or nutrition status of an organ corresponding a particular tissue type (e.g., liver cells). In yet another example, the determined characteristic can include a fraction of clinically-relevant DNA in a biological sample.

The comparison can be to a plurality of calibration values. The comparison can occur by inputting the first parameter into a calibration function fit to the calibration data that provides a change in the first parameter relative to a change in the characteristics in the sample. As another example, the one or more calibration values can correspond to other parameters in the one or more calibration samples.

Generally, it is preferred for the one or more calibration values determined from one or more calibration samples to be generated using a similar assay as used for the biological (test) sample. For example, a sequencing library can be generated in a same manner.

Process 2100 may include additional implementations, such as any single implementation or any combination of implementations described herein and/or in connection with one or more other processes described elsewhere herein or in US 2022/0010353 A1, the entire contents of which are incorporated herein by reference for all purposes.

Although FIG. 21 shows example blocks of process 2100, in some implementations, process 2100 may include additional blocks, fewer blocks, different blocks, or differently arranged blocks than those depicted in FIG. 21. Additionally, or alternatively, two or more of the blocks of process 2100 may be performed in parallel.

### IV. Fraction of Clinically-Relevant DNA

The results described in this document, including Section IIB, indicate the relationship between different jagged end types and the activity of different DNASEs. The 5′ protruding jagged has been revealed to correlate with the activity of DNASE1, and the blunt end has been revealed to relate with the activity of DFFB. As the expression of different DNASE varied in different tissues, the jagged end profiling may be used to deduce the tissue of origin of cfDNA.

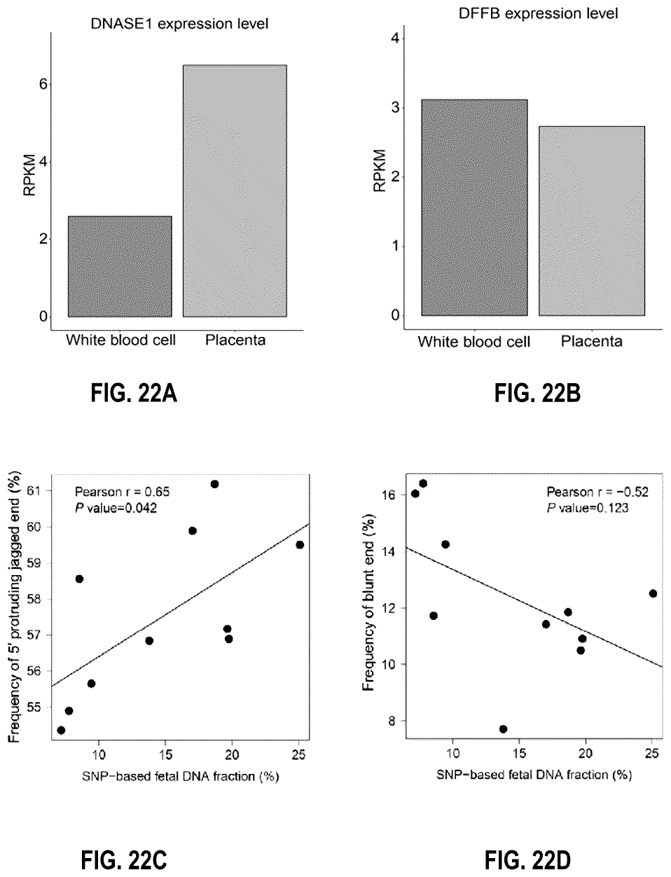

FIG. 22A is a graph of the mRNA expression level of DNASE1 in white blood cell and placenta. The y-axis shows RPKM, a normalized gene expression unit deduced from RNA sequencing results, i.e. reads per kilobase per million reads sequenced (Trapnell et al. Nat Biotechnol. 2010; 28:511-5). The x-axis shows white blood cells and placenta.

FIG. 22B is a graph of the mRNA expression level of DFFB in white blood cell and placenta. The axes are the same as FIG. 22A.

FIG. 22C is a graph of the correlation between fetal DNA fraction and the frequency of cfDNA fragments carrying 5′ protruding jagged ends. The x-axis shows the SNP-based fetal DNA fraction. The y-axis shows the frequency of 5′ protruding jagged ends.

FIG. 22D is a graph of the correlation between fetal DNA fraction and the frequency of cfDNA fragments carrying blunt ends. The x-axis shows the SNP-based fetal DNA fraction. The y-axis shows the frequency of blunt ends.

As shown in the graphs, placenta tissue showed higher expression of DNASE1 (FIG. 22A) and lower expression of DFFB (FIG. 22B) than white blood cells. Higher DNASE1 correlated with a higher 5′ protruding jagged end, and lower DFFB correlated with a lower blunt end of the fragment from the placenta origin. The frequency of 5′ protruding jagged end and blunt end could be used to reflect the fetal DNA fraction. FIG. 22C shows the frequency of 5′ protruding jagged end positively correlated with the fetal DNA fraction, while FIG. 22D shows the frequency of blunt end negatively correlated with the fetal DNA. This further suggests that the jagged end pattern of plasma DNA may reflect the tissue of origin of those molecules.

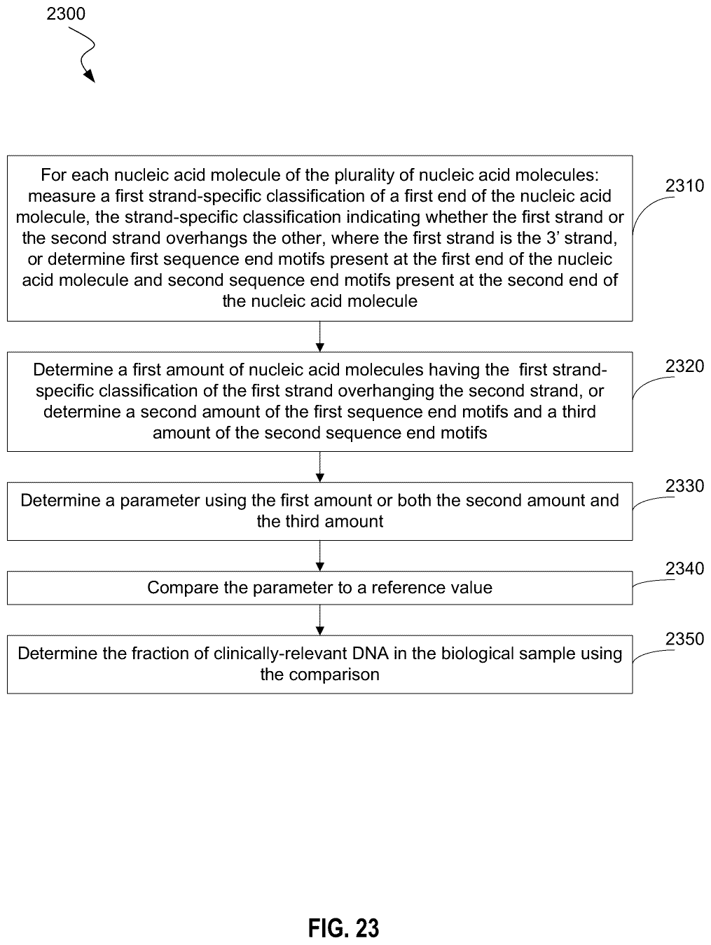

FIG. 23 is a flowchart of an example process 2300 for determining a fraction of clinically-relevant DNA in a biological sample. The biological sample may include a plurality of nucleic acid molecules that are cell-free. Each nucleic acid molecule of the plurality of nucleic acid molecules may be double-stranded with a first strand and a second strand. The biological sample may be obtained from an individual. At least some of the nucleic acid molecules of the plurality of nucleic acid molecules having nucleotides on one strand that have no complementary portion on the other strand. The biological sample may be any biological sample described herein. In some implementations, one or more process blocks of FIG. 23 may be performed by a system 2400.

The clinically-relevant DNA may be fetal DNA, tumor DNA, or DNA from a tissue type. The tissue type may include placenta, liver, white blood cells, colon, kidney, lung, or any other tissue type described herein.

At block 2310, at least two different steps are possible. First, a first strand-specific classification of a first end of the nucleic acid molecule may be measured for each nucleic acid molecule of the plurality of nucleic acid molecules. A strand-specific classification may indicate whether the first strand or the second strand overhangs the other, where the first strand is the 3′ strand. Second, first sequence end motifs present at the first end of the nucleic acid molecule and second sequence end motifs present at the second end of the nucleic acid molecule may be determined for each nucleic acid molecule of the plurality of nucleic acid molecules. The sequence end motifs may be of overhangs and/or of blunt ends.

At block 2320, a first amount of nucleic acid molecules having the first strand-specific classification of the first strand overhanging the second strand may be determined, or a second amount of the first sequence end motifs and a third amount of the second sequence end motifs may be determined.

At block 2330, a parameter using the first amount or both the second amount and the third amount may be determined. For example, the process may include determining the first amount, and determining the parameter may use the first amount. As another example, the process may include determining the second amount and the third amount, where determining the parameters uses both the second amount and the third amount.

In some embodiments, the amount of 5′ protruding ends may be used in addition to the amount of 3′ protruding ends. For example, process 2300 further includes determining the first amount, and determining a fourth amount of nucleic acid molecules having the same first strand-specific classification of the second strand overhanging the first strand, where determining the parameter uses the first amount and the fourth amount.

In some embodiments, the amount of blunt ends may be used in addition to the amount of 3′ protruding ends and/or the amount of blunt ends. For example, process 2300 further includes determining a fifth amount of nucleic acid molecules having the same first strand-specific classification of the first strand being even with the second strand, where determining the parameter uses the first amount, the fourth amount of molecules having the second strand overhang the first strand, and the fifth amount.

In some embodiments, the protruding ends at both ends of the nucleic acid molecules may be used. For example, process 2300 may further include for each nucleic acid molecule of the plurality of nucleic acid molecules, measuring a second strand-specific classification of the second end of the nucleic acid molecule. The process may further include determining a fourth amount of nucleic acid molecules having the same second strand-specific classification, where determining the parameter uses the first amount and the fourth amount.

In some embodiments, the first strand-specific classification, the first sequence end motifs, and the second sequence end motifs may be used. For example, the process may include determining the first amount, the second amount, and the third amount. Determining the parameter may further include using the first amount the second amount, and the third amount.

In some embodiments, the parameter may include a vector of the amounts. The vector may include elements of any vectors described herein, including the elements specifying the different combinations over overhangs. Determining the parameter may include using an amount other than the specific amounts mentioned. For example, the parameter may be a ratio or difference with an amount of all nucleic acid molecules.

At block 2340, the parameter may be compared to a reference value. The comparison may be performed similar to any comparison described herein, including block 1508. The reference value may be a value determined from one or more control samples having a known fraction of clinically-relevant DNA. A machine learning model may be used to perform the comparing of the parameter and the reference value. The machine learning model may include linear regression, logistic regression, deep recurrent neural network, Bayes classifier, hidden Markov model (HIMM), linear discriminant analysis (LDA), k-means clustering, density-based spatial clustering of applications with noise (DBSCAN), random forest algorithm, or support vector machine (SVM).

At block 2350, the fraction of clinically-relevant DNA in the biological sample may be determined using the comparison. If the parameter is statistically the same as the reverence value, then the level of the condition may be determined to be the same as the subject or subjects associated with the reference value.

In some embodiments, a first nuclease may be identified as differentially regulated in a target tissue type relative to at least one other tissue type of the plurality of tissue types. The clinically-relevant DNA molecules can be from the target tissue type. For example, DNASE1 expression is relatively upregulated in placental tissue compared with the DNASE1 expression level of white blood cells (FIG. 22A). In another example, DNASE1L3 expression is relatively downregulated in HCC cells compared with liver tissues in healthy subjects.

The first nuclease may be determined to preferentially cut DNA into DNA molecules that have a certain strand-specific classification and/or sequence end motif. In some instances, the cutting preference of the first nuclease is determined by analyzing a biological sample of another organism (e.g., mice). These strand-specific classifications and/or sequence end motifs may then be used to determine the fraction of clinically-relevant DNA.

Process 2300 may include additional implementations, such as any single implementation or any combination of implementations described below and/or in connection with one or more other processes described elsewhere herein. In a first implementation, the reference value is determined from one or more calibration samples whose fractional concentrations of the clinically-relevant DNA molecules are known.

Although FIG. 23 shows example blocks of process 2300, in some implementations, process 2300 may include additional blocks, fewer blocks, different blocks, or differently arranged blocks than those depicted in FIG. 23. Additionally, or alternatively, two or more of the blocks of process 2300 may be performed in parallel.

### V. Treatment

### A. Further Screening Modalities

Based on any classification, e.g., regarding a pathology or fractional concentration of clinically-relevant DNA, the subject can be referred for additional screening modalities, e.g. using chest X ray, ultrasound, computed tomography, magnetic resonance imaging, or positron emission tomography. Such screening may be performed for cancer.

### B. Treatment Selection

Embodiments of the present disclosure can accurately predict disease relapse (e.g., an increase in tumor DNA fraction following a decrease, classification of cancer existing after classification of cancer not existing), thereby facilitating early intervention and selection of appropriate treatments to improve disease outcome and overall survival rates of subjects. For example, an intensified chemotherapy can be selected for subjects, in the event their corresponding samples are predictive of disease relapse. In another example, a biological sample of a subject who had completed an initial treatment can be sequenced to identify viral DNA that is predictive of disease relapse. In such example, alternative treatment regimen (e.g., a higher dose) and/or a different treatment can be selected for the subject, as the subject's cancer may have been resistant to the initial treatment.

The embodiments may also include treating the subject in response to determining a classification of relapse of the pathology. For example, if the prediction corresponds to a loco-regional failure, surgery can be selected as a possible treatment. In another example, if the prediction corresponds to a distant metastasis, chemotherapy can be additionally selected as a possible treatment. In some embodiments, the treatment includes surgery, radiation therapy, chemotherapy, immunotherapy, targeted therapy, hormone therapy, stem cell therapy, or precision medicine. Based on the determined classification of relapse, a treatment plan can be developed to decrease the risk of harm to the subject and increase overall survival rate. Embodiments may further include treating the subject according to the treatment plan.

### C. Types of Treatments

Embodiments may further include treating the pathology in the patient after determining a classification for the subject. Treatment can be provided according to a determined level of pathology, the fractional concentration of clinically-relevant DNA, or a tissue of origin. For example, an identified mutation can be targeted with a particular drug or chemotherapy. The tissue of origin can be used to guide a surgery or any other form of treatment. And, the level of the pathology can be used to determine how aggressive to be with any type of treatment, which may also be determined based on the level of pathology. A pathology (e.g., cancer) may be treated by chemotherapy, drugs, diet, therapy, and/or surgery. In some embodiments, the more the value of a parameter (e.g., amount or size) exceeds the reference value, the more aggressive the treatment may be.

Treatment may include resection. For bladder cancer, treatments may include transurethral bladder tumor resection (TURBT). This procedure is used for diagnosis, staging and treatment. During TURBT, a surgeon inserts a cystoscope through the urethra into the bladder. The tumor is then removed using a tool with a small wire loop, a laser, or high-energy electricity. For patients with non-muscle invasive bladder cancer (NMIBC), TURBT may be used for treating or eliminating the cancer. Another treatment may include radical cystectomy and lymph node dissection. Radical cystectomy is the removal of the whole bladder and possibly surrounding tissues and organs. Treatment may also include urinary diversion. Urinary diversion is when a physician creates a new path for urine to pass out of the body when the bladder is removed as part of treatment.

Treatment may include chemotherapy, which is the use of drugs to destroy cancer cells, usually by keeping the cancer cells from growing and dividing. The drugs may involve, for example but are not limited to, mitomycin-C (available as a generic drug), gemcitabine (Gemzar), and thiotepa (Tepadina) for intravesical chemotherapy. The systemic chemotherapy may involve, for example but not limited to, cisplatin gemcitabine, methotrexate (Rheumatrex, Trexall), vinblastine (Velban), doxorubicin, and cisplatin.

In some embodiments, treatment may include immunotherapy. Immunotherapy may include immune checkpoint inhibitors that block a protein called PD-1. Inhibitors may include but are not limited to atezolizumab (Tecentriq), nivolumab (Opdivo), avelumab (Bavencio), durvalumab (Imfinzi), and pembrolizumab (Keytruda).

Treatment embodiments may also include targeted therapy. Targeted therapy is a treatment that targets the cancer's specific genes and/or proteins that contributes to cancer growth and survival. For example, erdafitinib is a drug given orally that is approved to treat people with locally advanced or metastatic urothelial carcinoma with FGFR3 or FGFR2 genetic mutations that has continued to grow or spread of cancer cells.

Some treatments may include radiation therapy. Radiation therapy is the use of high-energy x-rays or other particles to destroy cancer cells. In addition to each individual treatment, combinations of these treatments described herein may be used. In some embodiments, when the value of the parameter exceeds a threshold value, which itself exceeds a reference value, a combination of the treatments may be used. Information on treatments in the references are incorporated herein by reference.

### VI. Systems

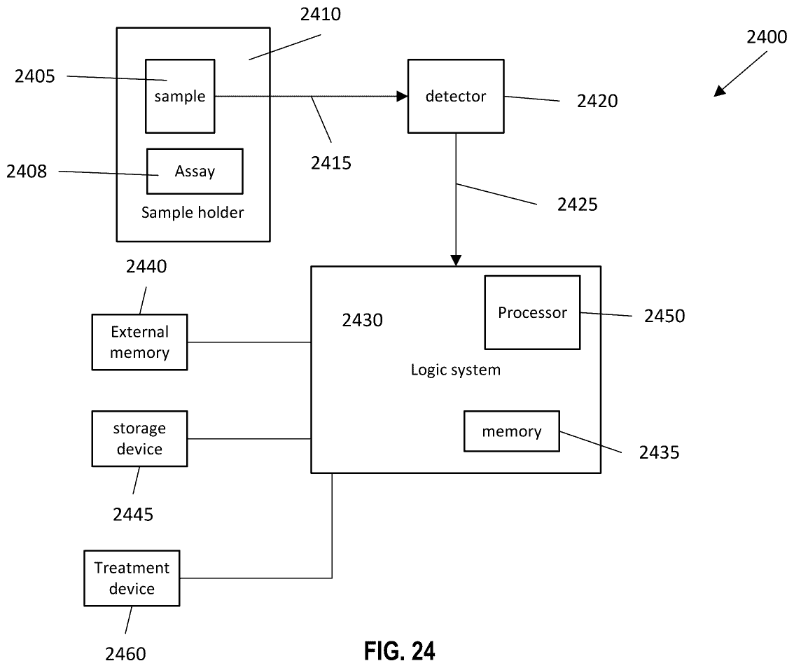

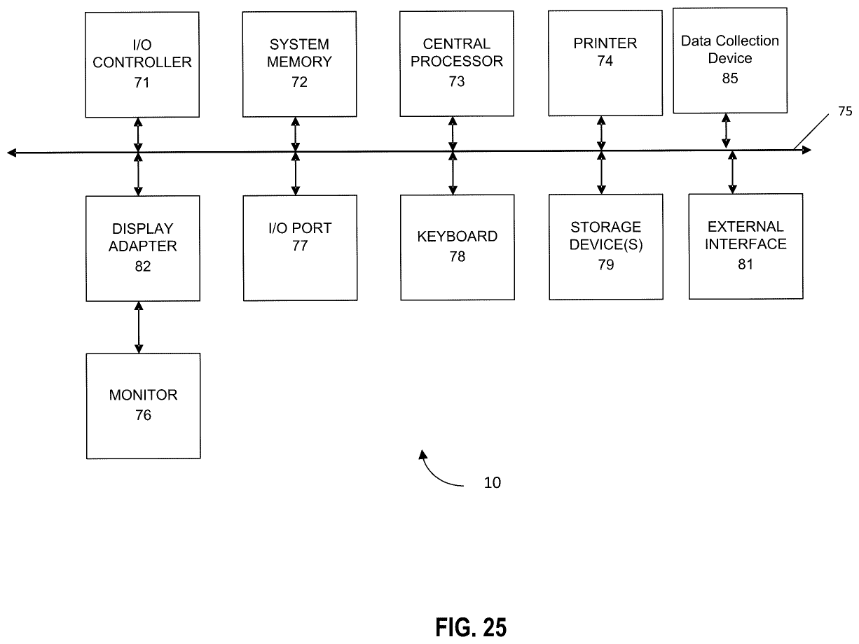

FIG. 24 illustrates a measurement system 2400 according to an embodiment of the present disclosure. The system as shown includes a biological object 2405, such as a biological sample of an organism (e.g., human), within an analysis device 2410, where an emitter 2408 can send waves to biological object 2405. For example, biological object 2405 can receive magnetic fields and/or radio waves from emitter 2408 to provide a signal of a physical characteristic 2415. Biological object 2405 may include objects treated with enzymes, labels, or primers or other agent to facilitate detection. An example of an analysis device can be a sequencing device. Analysis device 2410 may include multiple modules.

Physical characteristic 2415 (e.g., an optical intensity, a voltage, or a current), from the biological object is detected by detector 2420. Detector 2420 can take a measurement at intervals (e.g., periodic intervals) to obtain data points that make up a data signal. In one embodiment, an analog-to-digital converter converts an analog signal from the detector into digital form at a plurality of times. Analysis device 2410 and detector 2420 can form an assay system, e.g., a sequencing system that acquires data according to embodiments described herein. A data signal 2425 is sent from detector 2420 to logic system 2430. As an example, data signal 2425 can be used to determine identities of nucleotides in a biological object. Data signal 2425 can include various measurements made at a same time, e.g., different signals for different areas of biological object 2405, and thus data signal 2425 can correspond to multiple signals. Data signal 2425 may be stored in a local memory 2435, an external memory 2440, or a storage device 2445.

Logic system 2430 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 2430 and the other components may be part of a stand-alone or network connected computer system, or they may be directly attached to or incorporated in a device (e.g., an imaging system) that includes detector 2420 and/or analysis device 2410. Logic system 2430 may also include software that executes in a processor 2450. Logic system 2430 may include a computer readable medium storing instructions for controlling measurement system 2400 to perform any of the methods described herein. For example, logic system 2430 can provide commands to a system that includes analysis device 2410 such that magnetic emission or other physical operations are performed.

Measurement system 2400 may also include a treatment device 2460, which can provide a treatment to the subject. Treatment device 2460 can determine a treatment and/or be used to perform a treatment. Examples of such treatment can include surgery, radiation therapy, chemotherapy, immunotherapy, targeted therapy, hormone therapy, stem cell transplant, and implantation of radioactive seeds. Logic system 2430 may be connected to treatment device 2460, e.g., to provide results of a method described herein. The treatment device may receive inputs from other devices, such as an imaging device and user inputs (e.g., to control the treatment, such as controls over a robotic system).

Any of the computer systems mentioned herein may utilize any suitable number of subsystems. Examples of such subsystems are shown in FIG. 14 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 14 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, Lightning). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software with a generally programmable processor in a modular or integrated manner. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C #, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive, or an optical medium such as a compact disk (CD) or DVD (digital versatile disk) or Blu-ray disk, flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order that is logically possible. Additionally, portions of these steps may be used with portions of other steps from other methods. Also, all or portions of a step may be optional. Additionally, any of the steps of any of the methods can be performed with modules, units, circuits, or other means of a system for performing these steps.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The above description of example embodiments of the present disclosure has been presented for the purposes of illustration and description and are set forth so as to provide those of ordinary skill in the art with a complete disclosure and description of how to make and use embodiments of the present disclosure. It is not intended to be exhaustive or to limit the disclosure to the precise form described nor are they intended to represent that the experiments are all or the only experiments performed. Although the disclosure has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the disclosure being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

All patents, patent applications, publications, and descriptions mentioned herein are hereby incorporated by reference in their entirety for all purposes as if each individual publication or patent were specifically and individually indicated to be incorporated by reference and are incorporated herein by reference to disclose and describe the methods and/or materials in connection with which the publications are cited. None is admitted to be prior art.

1. A method of analyzing a biological sample comprising a plurality of nucleic acid molecules that are cell-free, the method comprising:
for each nucleic acid molecule of the plurality of nucleic acid molecules:
ligating a first hairpin adapter to a first strand of the nucleic acid molecule and a second strand of the nucleic acid molecule at a first end of the nucleic acid molecule, wherein the first hairpin adapter comprises a first sequence identifier that identifies a first length of zero or more nucleotides at a first terminus of the first hairpin adapter having no complementary portion at a second terminus of the first hairpin adapter, and
ligating a second hairpin adapter to the first strand and the second strand at a second end of the nucleic acid molecule, wherein the second hairpin adapter comprises a second sequence identifier that identifies a second length of zero or more nucleotides at a first terminus of the second hairpin adapter having no complementary portion at a second terminus of the first hairpin adapter, thereby generating a plurality of ligated nucleic acid molecules;
performing rolling circle amplification on a first subset of the plurality of ligated nucleic acid molecules to form a plurality of concatemers; and
sequencing each concatemer of the plurality of concatemers to identify the respective first sequence identifier and the respective second sequence identifier.

2. The method of claim 1, wherein sequencing occurs simultaneously with performing rolling circle amplification.

3. The method of claim 1, further comprising:
determining first lengths of overhangs present at the first ends of nucleic acid molecules of the first subset of the plurality of nucleic acid molecules using the first sequence identifiers, and
determining second overhangs present at the second ends of nucleic acid molecules of the first subset of the plurality of nucleic acid molecules using the second sequence identifiers.

4. The method of claim 1, further comprising:
adding exonucleases to the plurality of ligated nucleic acid molecules to remove a second subset of the plurality of ligated nucleic acid molecules, wherein:
the first subset of the plurality of nucleic acid molecules does not include any of the nucleic acid molecules in the second subset,
for each nucleic acid molecule of the second subset, either:
the respective nucleic acid molecule is not completely hybridized to the respective first hairpin adapter or the respective second hairpin adapter, or
the respective first hairpin adapter or the respective second hairpin adapter is not completely hybridized to the respective nucleic acid molecule.

5. The method of claim 1, further comprising:
determining first sequence end motifs present at the first ends of nucleic acid molecules of the first subset of the plurality of nucleic acid molecules using the first sequence identifiers; and
determining second sequence end motifs present at the second ends of nucleic acid molecules of the first subset of the plurality of nucleic acid molecules using the second sequence identifiers.

6. The method of claim 1, further comprising:
determining whether a 5′ strand or a 3′ strand overhangs the other for each nucleic acid molecule having an overhang at the first end of the first subset using the respective first sequence identifier, and
determining whether a 5′ strand or a 3′ strand overhangs the other for each nucleic acid molecule having an overhang at the second end of the first subset using the respective second sequence identifier.

7. The method of claim 1, wherein:
the biological sample is obtained from a female subject pregnant with a fetus,
the method further comprising:
selecting reads corresponding to a subset of the plurality of nucleic acid molecules having the 5′ strand or the 3′ strand overhanging the other end, and
analyzing the subset of nucleic acid molecules for a characteristic of the fetus.

8. The method of claim 1, wherein:
each first hairpin adapter of a plurality of first hairpin adapters comprises a first cleavage site, and
each second hairpin adapter of a plurality of second hairpin adapters comprises a second cleavage site,
the method further comprising:
cleaving each concatemer of the plurality of concatemers at a respective first cleavage site and at a respective second cleavage site.

9. The method of claim 1, wherein each nucleic acid molecule of a second portion of the first subset has the respective first strand even with the respective second strand at the respective first end.

10. The method of claim 1, wherein:
each nucleic acid molecule of a second portion of the first subset has the respective second strand overhanging the respective first strand at the respective first end,
the respective first strand is the 5′ strand, and
the respective second strand is the 3′ strand.

11. A method of analyzing a biological sample comprising a plurality of nucleic acid molecules that are cell-free, the method comprising:
for each nucleic acid molecule of the plurality of nucleic acid molecules:
ligating a first hairpin adapter to a first strand of the nucleic acid molecule and a second strand of the nucleic acid molecule at a first end of the nucleic acid molecule, wherein the first hairpin adapter comprises a first sequence identifier that identifies a first length of zero or more nucleotides at a first terminus of the first hairpin adapter having no complementary portion at a second terminus of the first hairpin adapter, and
ligating a second hairpin adapter to the first strand and the second strand at a second end of the nucleic acid molecule, wherein the second hairpin adapter comprises a second sequence identifier that identifies a first length of zero or more nucleotides at a first terminus of the second hairpin adapter having no complementary portion at a second terminus of the first hairpin adapter, thereby generating a plurality of ligated nucleic acid molecules;
adding exonucleases to the plurality of ligated nucleic acid molecules to remove a first subset of the plurality of ligated nucleic acid molecules, wherein:
for each nucleic acid molecule of the first subset, either:
the respective nucleic acid molecule is not completely hybridized to the respective first hairpin adapter or the respective second hairpin adapter, or
the respective first hairpin adapter or the respective second hairpin adapter is not completely hybridized to the respective nucleic acid molecule,
sequencing each ligated nucleic acid molecule of a second subset of the plurality of ligated nucleic acid molecules to identify the respective first sequence identifier and the respective second sequence identifier, wherein the second subset remains in the biological sample after removing the first subset.

12. The method of claim 11, further comprising:
determining first lengths of overhangs present at the first ends of nucleic acid molecules of the second subset of the plurality of nucleic acid molecules using the first sequence identifiers, and
determining second overhangs present at the second ends of nucleic acid molecules of the second subset of the plurality of nucleic acid molecules using the second sequence identifiers.

13. The method of claim 11, further comprising:
determining first sequence end motifs of overhangs present at the first ends of nucleic acid molecules of the second subset of the plurality of nucleic acid molecules using the first sequence identifiers; and
determining second sequence end motifs of overhangs present at the second ends of nucleic acid molecules of the second subset of the plurality of nucleic acid molecules using the second sequence identifiers.

14. The method of claim 11, further comprising:
determining whether a 5′ strand or a 3′ strand overhangs the other for each nucleic acid molecule having an overhang at the first end of the second subset using the respective first sequence identifier, and
determining whether a 5′ strand or a 3′ strand overhangs the other for each nucleic acid molecule having an overhang at the second end of the second subset using the respective second sequence identifier.

15. The method of claim 11, wherein:
the biological sample is obtained from a female subject pregnant with a fetus,
the method further comprising:
selecting reads corresponding to a subset of a plurality of nucleic acid molecules having the 5′ strand or the 3′ strand overhanging the other end, and
analyzing the subset of nucleic acid molecules for a characteristic of the fetus.

16. A method of analyzing a biological sample comprising a plurality of nucleic acid molecules that are cell-free, each nucleic acid molecule of the plurality of nucleic acid molecules being double-stranded with a first strand and a second strand, the biological sample being obtained from an individual, at least some of the nucleic acid molecules of the plurality of nucleic acid molecules having nucleotides on one strand that have no complementary portion on the other strand, the method comprising:
for each nucleic acid molecule of the plurality of nucleic acid molecules:
measuring a first strand-specific classification of a property of a first end of the nucleic acid molecule, a strand-specific classification indicating whether the first strand or the second strand overhangs the other;
determining a jagged end value using the first strand-specific classifications of the plurality of nucleic acid molecules;
comparing the jagged end value to a reference value; and
determining a level of a condition of the individual using the comparison.

17. The method of claim 16, further comprising:
for each nucleic acid molecule of the plurality of nucleic acid molecules:
measuring a second strand-specific classification of the second end of the nucleic acid molecule,
wherein:
determining the jagged end value uses the second strand-specific classifications.

18. The method of claim 16, the strand-specific classification further indicates the length of an overhang.

19. The method of claim 17, wherein:
the jagged end value is an element in a vector,
the vector includes a plurality of elements, and
the plurality of elements includes amounts of nucleic acid molecules in the following classifications:
blunt-ended at the first end and blunt-ended at the second end,
5′ overhang at the first end and blunt-ended at the second end,
3′ overhang at the first end and blunt-ended at the second end,
5′ overhang at the first end and 3′ overhang at the second end,
5′ overhang at the first end and 5′ overhang at the second end, and
3′ overhang at the first end and 3′ overhang at the second end;
the method further comprising:
comparing the vector to a reference vector;
wherein determining the level of the condition of the individual uses the comparison of the vector to the reference vector.

20. The method of claim 19, wherein the plurality of elements comprises a classification of nucleic acid molecules having sizes in one or more size ranges.

21. The method of claim 16, wherein the condition is nuclease activity deficiency.

22. A method of analyzing a biological sample comprising a plurality of nucleic acid molecules, each nucleic acid molecule of the plurality of nucleic acid molecules being double-stranded with a first strand and a second strand, at least some of the nucleic acid molecules of the plurality of nucleic acid molecules having nucleotides on one strand that have no complementary portion on the other strand, the biological sample being obtained from an individual, the method comprising:
for each nucleic acid molecule of the plurality of nucleic acid molecules:
determining a first sequence end motif of the first strand at a first end of the nucleic acid molecule, and
determining a second sequence end motif of the second strand at the first end of the nucleic acid molecule;
determining a first amount of nucleic acid molecules having a first combination of the first sequence end motif and the second sequence end motif at the first end;
generating a value of an end motif parameter using the first amount;
comparing the value of the end motif parameter to a reference value; and
determining a level of a condition of the individual using the comparison.

23. The method of claim 22, wherein the first strand has 3′ end at the first end, and the first strand overhangs the second strand.

24. The method of claim 22, wherein the first end comprises a blunt end.

25. The method of claim 22, further comprising:
determining a second amount of nucleic acid molecules having a second combination of a third sequence end motif and a fourth sequence end motif at the first end,
wherein:
generating the value of the end motif parameter uses the second amount.

26. The method of claim 22, wherein:
the plurality of nucleic acid molecules is a first plurality of nucleic acid molecules,
the biological sample comprises a second plurality of nucleic acid molecules, and
the first plurality of nucleic acid molecules includes a subset of the second plurality of nucleic acid molecules,
the method further comprising:
for each nucleic acid molecule of a second plurality of nucleic acid molecules:
determining a third sequence end motif on the first strand at a second end of the nucleic acid molecule, and
determining a fourth sequence end motif on the second strand at the second end of the nucleic acid molecule,
determining a second amount of nucleic acid molecules having a second combination of the third sequence end motif and the fourth sequence end motif at the second end, wherein:
generating the value of the end motif parameter uses the second amount.

27. The method of claim 22, further comprising:
for each nucleic acid molecule of the plurality of nucleic acid molecules:
measuring a first strand-specific classification of a first end of the nucleic acid molecule, a strand-specific classification indicating whether the first strand or the second strand overhangs the other,
wherein determining the first amount comprises determining the first amount of nucleic acid molecules having the first combination and the first strand-specific classification.

28. The method of claim 27, further comprising:
for each nucleic acid molecule of the plurality of nucleic acid molecules:
measuring a second strand-specific classification of the second end of the nucleic acid molecule,
wherein determining the first amount comprises determining the first amount of nucleic acid molecules having the first combination, the first strand-specific classification, and the second strand-specific classification.

29. The method of claim 22, further comprising:
for each nucleic acid molecule of the plurality of nucleic acid molecules:
determining a third sequence end motif of the first strand at a second end of the nucleic acid molecule, and
determining a fourth sequence end motif on the second strand at the second end of the nucleic acid molecule;
wherein:
the first combination is of the first sequence end motif at the first end, the second sequence end motif at the first end, the third sequence end motif at the second end, and the fourth sequence end motif at the second end.

30. The method of claim 29, further comprising:
for each nucleic acid molecule of the plurality of nucleic acid molecules:
measuring a first strand-specific classification of a first end of the nucleic acid molecule, the strand-specific classification indicating whether the first strand or the second strand overhangs the other,
measuring a second strand-specific classification of the second end of the nucleic acid molecule,
wherein determining the first amount comprises determining the first amount of nucleic acid molecules having the first combination, the first strand-specific classification, and the second strand-specific classification.

31. The method of claim 22, wherein the condition is cancer.

32. The method of claim 22, wherein the condition is a nuclease activity deficiency.

33-75. (canceled)
